(19) United States
(12) Patent Application Publication
(10) Pub. No.: US 2025/0277037 A1
(43) Pub. Date: Sep. 4, 2025

(54) ANTIBODIES SPECIFIC TO PD-1 AND METHODS OF USE

(71) Applicant: UNIVERSITY OF UTAH RESEARCH FOUNDATION, Salt Lake City, UT (US)

(72) Inventors: Mingnan Chen, Salt Lake City, UT (US); Shuyun Dong, Salt Lake City, UT (US)

(21) Appl. No.: 18/857,990

(22) PCT Filed: Apr. 20, 2023
(86) PCT No.: PCT/US2023/066037

§ 371 Date: Oct. 18, 2024

Related U.S. Application Data
(60) Provisional application No. 63/332,940, filed on Apr. 20, 2022.

(51) Int. Cl. C07K 16/28; A61K 39/00; A61P 3/10; A61P 35/00

(52) U.S. Cl. CPC C07K 16/2818; A61P 3/10; A61P 35/00; A61K 2039/505; C07K 2317/734; C07K 2317/76; C07K 2317/77; C07K 2317/92

(57) ABSTRACT

Disclosed herein are anti-PD-1 antibodies useful in treating autoimmune diseases, treating cancer, treating metastatic cancer or preventing cancer metastasis in a subject. Further disclosed herein are anti-PD1 antibodies useful in depleting PD-1 positive cells.

### CROSS REFERENCE TO RELATED APPLICATIONS

This application claims the benefit of the filing date of U.S. Provisional Application No. 63/332,940, filed on Apr. 20, 2022. The content of this earlier filed application is hereby incorporated by reference in its entirety.

### REFERENCE TO A SEQUENCE LISTING

The Sequence Listing submitted herein as a text file named “21101_0437P1_SL.” created on Apr. 18, 2023, and having a size of 16,384 bytes is hereby incorporated by reference pursuant to 37 C.F.R. § 1.52 (e) (5).

### BACKGROUND

Programmed death-1 (PD-1) (also known as CD279) is a receptor expressed on the T cells that acts to down-regulate the immune system by abrogating T cell receptor-induced signals and preventing antigen-mediated T cell activation. The interaction between PD-1 and its ligand (programmed death-ligand 1, PD-L1) plays a role in maintaining self-tolerance and avoiding autoimmune diseases. However, PD-1/PD-L1 could also prevent the activation of T cells in the tumor and thus result in immune resistance.

Despite the clinical benefits, therapy resistance remains a significant challenge for the further application of PD-1/PD-L1 blockade therapy. It is estimated that a minority of patients experience a positive response to PD-1/PD-L1 blockade therapy, and the resistance might lead to cancer progression in patients with clinical response. To overcome the limitation in therapy resistance, improved anti-PD-1/PD-L1 based immunotherapy strategies are needed.

### SUMMARY

Disclosed herein are isolated antibodies comprising a light chain variable region and a heavy chain variable region, wherein the light chain variable region comprises a complementarity determining region light chain 1 (CDRL1) amino acid sequence of SEQ ID NO: 4; a complementarity determining region light chain 2 (CDRL2) amino acid sequence of SEQ ID NO: 5; and a complementarity determining region light chain 3 (CDRL3) amino acid sequence of SEQ ID NO: 6; and wherein the heavy chain variable region comprises a complementarity determining region heavy chain 1 (CDRH1) amino acid sequence of SEQ ID NO: 1; a complementarity determining region heavy chain 2 (CDRH2) amino acid sequence of SEQ ID NO: 2; and a complementarity determining region heavy chain 3 (CDRH3) amino acid sequence of SEQ ID NO: 3.

Disclosed herein are isolated antibodies comprising a light chain variable region amino acid sequence of SEQ ID NO: 8, 10, 13, 14 or 15 and a heavy chain variable region amino acid sequence of SEQ ID NO: 7, 9, 11, or 12.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 4; a determining region light chain 2 (CDRL2) amino acid sequence of SEQ ID NO: 5; and a determining region light chain 3 (CDRL3) amino acid sequence of SEQ ID NO: 6; and wherein the heavy chain variable region comprises a complementarity determining region heavy chain 1 (CDRH1) amino acid sequence of SEQ ID NO: 1; a complementarity determining region heavy chain 2 (CDRH2) amino acid sequence of SEQ ID NO: 2; and a complementarity determining region heavy chain 3 (CDRH3) amino acid sequence of SEQ ID NO: 3, wherein one or more of the CDRL1, CDRL2. CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 8, 10, 13, 14 or 15 and a heavy chain variable region amino acid sequence of SEQ ID NO: 7, 9, 11, or 12, wherein the isolated antibody comprises 1, 2, 3, 4, or 5 conservative amino acid substitutions in the light or heavy chain variable region amino acid sequences.

### BRIEF DESCRIPTION OF THE DRAWINGS

FIGS. 1A-D show the design and generation of D-αPD-1. FIG. 1A is a schematic showing the design of D-αPD-1. Black and dark gray fragments are expressed by the heavy chain plasmid, and light gray fragments are expressed by the light chain plasmid. FIG. 1B shows the agarose gel image of plasmid digestion products from the plasmid encoding heavy chain (lane 1) and the plasmid encoding light chain (lane 2). Heavy chain plasmid DNA was digested using XbaI and EcoRV. Light chain plasmid DNA was digested using SacI and EcoRV. The upper bands of each lane represent cleaved vectors: the lower bands of each lane represent the coding genes of heavy chain and light chain, respectively. FIG. 1C shows the non-reducing SDS-PAGE image of D-αPD-1 (lane 1) after purification, compared with B-αPD-1 (lane 2) and commercial IgG2a (lane 3). FIG. 1D shows the reducing SDS-PAGE gel of D-αPD-1 (lane 1) compared with B-αPD-1 (lane 2) and commercial IgG2a (lane3). The upper band represents the heavy chain; the lower band shows the light chain.

FIGS. 2A-B show the interactions between D-αPD-1 and EL4 cells. FIG. 2A shows the mean fluorescence intensity (MFI) of EL4 cells after the cells were incubated with D-αPD-1 or mouse IgG2a at different concentrations on ice for 30 min, then were stained with PE-anti-mouse-IgG2a Ab and analyzed by flow cytometry. FIG. 2B show the MFI of EL4 cells after the cells were incubated with Alexa-647-labeled D-αPD-1 at different concentrations at 4° C. or 37° C. for 1 hour. The data are presented as means±SD of each treatment (N=3). (****P<0.0001)

FIGS. 3A-D show in vivo depletion of PD-1+ cells by D-αPD-1. FIG. 3A shows the percentage of PD-1⁺ cells in T cells from bone marrow of mice which were inoculated EL4 cells and then treated with D-αPD-1, B-αPD-1, or IgG2a. The mice were sacrificed 10 days after inoculation. The data are presented as mean percentage±SD of each treatment (N=5, unpaired two-sided t-test). FIG. 3B show survival of C57/BL6 mice inoculated with EL4 cells followed by the i.p treatment of D-αPD-1, IgG2a or PBS. N=5. FIG. 3C shows the percentage of EL4 cells in T cells from blood of mice which were inoculated EL4 cells and then treated with D-αPD-1 or PBS. Endpoint means the humane endpoint for mice in PBS treated group. D-αPD-1 treated mice were euthanized and examined at the time matching the endpoints of PBS-treated mice. Each symbol represents the EL4 fraction value of one mouse. The presented values are the means of EL4 cell fractions of treatment groups. (N=4˜5, unpaired two-sided T-test). FIG. 3D show the percentage of EL4 cells in T cells from bone marrow of mice which were inoculated EL4 cells and then treated with D-αPD-1 or PBS. Endpoint means the humane endpoint for mice in PBS treated group. D-αPD-1 treated mice were euthanized and examined at the time matching the endpoints of PBS-treated mice. Each symbol represents the EL4 fraction value of one mouse. The presented values are the means of EL4 cell fractions of treatment groups. (N=4˜5, unpaired two-sided T-test). FIG. 3E shows the body weight change of mice with different treatments N=4˜5. FIG. 3F shows survival of C57/BL6 mice inoculated with PD-1 knock out EL4 cells followed by the i.p treatment of D-αPD-1 or PBS. N=5. FIG. 3G shows the median survival times of mice with different treatments. (*P<0.05; **P<0.01; ***P<0.001; ****P<0.0001; ns; not significant)

FIGS. 4A-B show CDC of D-αPD-1. FIG. 4A shows the viability of EL4 cells after the cells were cocultured with baby rabbit complement and different concentrations (μg/mL) of D-αPD-1 or IgG2a for 3 hrs. FIG. 4B shows the viability of EL4 (PD-1^{KO}) after the cells were cocultured with baby rabbit complement and different concentrations (μg/mL) of D-αPD-1 for 3 hrs. MTS assay was used to evaluate viability of the cells. Values represent the mean (±SD) percentage of live cells in all cells. N=5. (**P<0.01; ****P<0.0001; ns; not significant) FIGS. 5A-C shows ADCP of D-αPD-1. FIG. 5A shows FcγRIV binding of Raw 264.7 cells to different Alexa-647-labeled-antibodies at 4 μg/mL at 4° C. for 30 minutes. The presented data are MFIs of cells after incubation±SD (N=4). FIG. 5B shows inhibition effect of D-αPD-1 to the binding of anti-FcγRIV antibody with RAW 264.7 cells. The cells were treated with antibodies at different concentrations (μg/mL) at 4° C. for 30 mins before wash and stain with PE-anti-FcγRIV. The represented data are means of inhibition percentage±SD (N=5). FIG. 5C shows ADCP response towards PD-1+EL4 cells by Raw 264.7 cell in the presence of D-αPD-1 or isotype IgG2a. The data are presented as the mean percentage of phagocytosis±SD of each treatment (N=3). (*P<0.05; **P<0.01; ****P<0.0001)

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list. Further, the term “or” means “and/or” unless explicitly indicated to refer to alternatives only or the alternatives are mutually exclusive, although the disclosure supports a definition that refers to only alternatives and “and/or.”

As used herein, the term “another” means at least a second or more.

As used herein, the term “about” is used to indicate that a value includes the inherent variation of error for the device, the method being employed to determine the value, or the variation that exists among the study subjects.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered. As used herein, the term “subject” refers to either a human or a non-human animal, such as primates, mammals, and vertebrates having cancer or an autoimmune disease. In some aspects, the subject in need will or is predicted to benefit from anti-αPD-1 antibody treatment.

As used herein, the term “patient” refers to a subject afflicted with a disease or disorder. The term “patient” includes human and veterinary subjects. In some aspects of the disclosed methods, the “patient” has been diagnosed with a need for treatment for cancer or an autoimmune disease, such as, for example, prior to the administering step.

As used herein, the term “treat,” “treatment,” or “treating” refers to administration or application of a therapeutic agent to a subject in need thereof, or performance of a procedure or modality on a subject, for the purpose of obtaining at least one positive therapeutic effect or benefit, such as treating a disease or health-related condition. For example, a treatment can include administration of a pharmaceutically effective amount of an antibody, or a composition or formulation thereof that specifically binds to αPD-1 positive cells for the purpose of treating various autoimmune diseases or cancer. The terms “treatment regimen,” “dosing regimen,” or “dosing protocol,” are used interchangeably and refer to the timing and dose of a therapeutic agent, such as an anti-αPD-1 antibody as described herein.

As used herein, the term “therapeutic benefit” or “therapeutically effective” refers the promotion or enhancement of the well-being of a subject in need (e.g., a subject with cancer or an autoimmune disease) with respect to the medical treatment, therapy, dosage administration, of a condition, particularly as a result of the use of the anti-αPD-1 antibodies and the performance of the described methods. This includes, but is not limited to, a reduction in the frequency or severity of the signs or symptoms of a disease. In some aspects, treatment of cancer or metastatic cancer may involve, for instance, a reduction in the size of a tumor, a reduction in the invasiveness or severity of a tumor, a reduction infiltration of cancer cells into a peripheral tissue or organ; a reduction in the growth rate of the tumor or cancer, or the prevention or reduction of metastasis. Treatment of cancer may also refer to achieving a sustained response in a subject or prolonging the survival of a subject with cancer.

As used herein, the term “administer” or “administration” refers to the act of physically delivering, e.g., via injection or an oral route, a substance as it exists outside the body into a patient, such as by oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance typically occurs after the onset of the disease, disorder or condition or symptoms thereof. Prophylactic treatment involves the administration of the substance at a time prior to the onset of the disease, disorder or condition or symptoms thereof.

As used herein, the term “effective amount” refers to the quantity or amount of a therapeutic (e.g., an antibody or pharmaceutical composition provided herein) which is sufficient to reduce, diminish, alleviate, and/or ameliorate the severity and/or duration of a cancer or a symptom related thereto. This term also encompasses an amount necessary for the reduction or amelioration of the advancement or progression of a cancer or an autoimmune disease; the reduction or amelioration of the recurrence, development of a cancer or autoimmune disease; and/or the improvement or enhancement of the prophylactic or therapeutic effect(s) of another cancer or autoimmune therapy. In some aspects, the effective amount of an antibody provided herein is from about or equal to 0.1 mg/kg (mg of antibody per kg weight of the subject) to about or equal to 100 mg/kg. In some aspects, an effective amount of an antibody provided therein is about or equal to 0.1 mg/kg, about or equal to 0.5 mg/kg, about or equal to 1 mg/kg, about or equal to 3 mg/kg, about or equal to 5 mg/kg, about or equal to 10 mg/kg, about or equal to 15 mg/kg, about or equal to 20 mg/kg, about or equal to 25 mg/kg, about or equal to 30 mg/kg, about or equal to 35 mg/kg, about or equal to 40 mg/kg, about or equal to 45 mg/kg, about or equal to 50 mg/kg, about or equal to 60 mg/kg, about or equal to 70 mg/kg, 80 mg/kg, 90 mg/kg, or 100 mg/kg. These amounts are meant to include amounts and ranges therein. In some aspects, “effective amount” also refers to the amount of an antibody provided herein to achieve a specified result (e.g., binding to αPD-1 positive cells; or depleting αPD-1 positive cells).

The term “in combination” in the context of the administration of other therapies (e.g., other agents, cancer drugs, cancer therapies, immunosuppressants) includes the use of more than one therapy (e.g., drug therapy and/or cancer therapy and/or immunosuppressants). Administration “in combination with” one or more further therapeutic agents includes simultaneous (e.g., concurrent) and consecutive administration in any order. The use of the term “in combination” does not restrict the order in which therapies are administered to a subject. By way of nonlimiting example, a first therapy (e.g., agent, such as an anti-αPD-1 positive cells antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with cancer.

In some aspects, the second therapy or agent that can be used in combination with drugs used to treat cancer include but are not limited to chemotherapeutic agents, radiotherapy, immunotherapy, and surgery.

In some aspects, the second therapy or agent that can be used in combination with drugs used to treat multiple sclerosis include but are not limited to interferon beta, glatiramer acetate, and fingolimod.

In some aspects, the second therapy or agent that can be used in combination with drugs used to treat type 1 diabetes include but are not limited to symptom relieving or management agents, and insulin.

In some aspects, the second therapy or agent that can be used in combination with drugs used to autoimmune diseases include but are not limited for type-1 diabetes, symptom relieving or management agents and insulin; and for multiple sclerosis, physical therapy, muscle relaxants, and medications to reduce fatigue.

In some aspects, the combination of therapies (e.g., use of agents, including therapeutic agents) may be more effective than the additive effects of any two or more single therapy (e.g., have a synergistic effect). For example, a synergistic effect of a combination of therapeutic agents frequently permits the use of lower dosages of one or more of the agents and/or less frequent administration of the agents to a cancer patient. The ability to utilize lower dosages of therapeutics and cancer or autoimmune disease therapies and/or to administer the therapies less frequently reduces the potential for toxicity associated with the administration of the therapies to a subject without reducing the effectiveness of the therapies. In addition, a synergistic effect may result in improved efficacy of therapies in the treatment or alleviation of a cancer or an autoimmune disease. Also, a synergistic effect demonstrated by a combination of therapies (e.g., therapeutic agents) may avoid or reduce adverse or unwanted side effects associated with the use of any single therapy.

As used herein, the term “comprising” can include the aspects “consisting of” and “consisting essentially of.” “Comprising” can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, disorder, sickness, illness, complaint, affection. In some aspects, the disease or disorder or condition can be a cancer or metastatic cancer or an autoimmune disease. In some aspects, the cancer can be breast cancer, colon cancer, lymphatic system cancers, pancreatic cancer, lung cancer, skin cancer (e.g., melanoma), esophageal cancer, bladder cancer, head and neck cancers, and stomach cancer. In some aspects, the autoimmune disease can be multiple sclerosis, type-1 diabetes, systemic lupus erythematosus, or rheumatoid arthritis. In some aspects, the disease or disorder or condition can be any disease or disorder.

As used herein, the terms “anti-αPD-1 antibody”; “anti-PD-1 antibody 1”; D-αPD-1″; and “D-αPD-1” can be used interchangeable to refer to an antibody disclosed herein that binds to PD-1, a receptor present on cells.

Abbreviations for the amino acid residues that comprise polypeptides and peptides described herein, and conservative substitutions for these amino acid residues are shown in Table 1 below. A polypeptide that contains one or more conservative amino acid substitutions or a conservatively modified variant of a polypeptide described herein refers to a polypeptide in which the original or naturally occurring amino acids are substituted with other amino acids having similar characteristics, for example, similar charge, hydrophobicity/hydrophilicity, side-chain size, backbone conformation, structure and rigidity, etc. Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity.

TABLE 1
Amino Acid Residues and Examples of
Conservative Amino Acid Substitutions

| Original residue | |
|---|---|
| Three letter code and | Conservative |
| Single letter code | substitution(s) |
| Alanine (Ala) (A) | Gly; Ser |
| Arginine (Arg) (R) | Lys; His |
| Asparagine (Asn) (N) | Gln; His |
| Aspartic Acid (Asp) (D) | Glu: Asn |
| Cysteine (Cys) (C) | Ser; Ala |
| Glutamine (Gln) (Q) | Asn |
| Glutamic Acid (Glu) (E) | Asp; Gln |
| Glycine (Gly) (G) | Ala |
| Histidine (His) (H) | Asn; Gln |
| Isoleucine (Ile) (I) | Leu; Val |
| Leucine (Leu) (L) | Ile; Val |
| Lysine (Lys) (K) | Arg; His |
| Methionine (Met) (M) | Leu; Ile; Tyr |
| Phenylalanine (Phe) (F) | Tyr; Met; Leu |
| Proline (Pro) (P) | Ala |
| Serine (Ser) (S) | Thr |
| Threonine (Thr) (T) | Ser |
| Tryptophan (Trp) (W) | Tyr; Phe |
| Tyrosine (Tyr) (Y) | Trp; Phe |
| Valine (Val) (V) | Ile; Leu |

Programmed cell death protein 1, also known as PD-1 or CD279, is a protein on the surface of T and B cells that has a role in regulating the immune system's response to the cells of the human body by down-regulating the immune system and promoting self-tolerance by suppressing T cell inflammatory activity.

As used herein, the term “polypeptide” or “peptide” refers to a polymer of amino acids of three or more amino acids in a serial array, linked through peptide bonds. As used herein, the term “amino acid sequence” refers to a list of abbreviations, letters, characters or words representing amino acid residues. “Polypeptides” can be proteins, protein fragments, protein analogs, oligopeptides and the like. The amino acids that comprise the polypeptide may be naturally derived or synthetic. The polypeptide may be purified from a biological sample. For example, a PD-1 polypeptide or peptide may be composed of at least 3, 4, 5, 6, 7, 8, 9, 10, 11, 12, 13, 14, 15, 16, 17, 18, 19, 20, 21, 22, 23, 24, or 25 contiguous amino acids of human PD-1. In some aspects, the polypeptide has at least 25, 30, 35, 40, 45, 50, 55, 60, 65, 70, 75, 80, 85, 90, 95, 100, 105, 110, 115, 120, 125, 130, or 135, contiguous amino acids of human PD-1. In some aspects, the PD-1 polypeptide comprises at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 200 contiguous amino acid residues, at least 288 contiguous amino acid residues of the amino acid sequence of the PD-1 polypeptide. Human PD-1, Uniprot accession number, Q15116; Mouse PD-1, Uniprot accession number, Q02242.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

As used herein, the term “analog” refers to a polypeptide that possesses a similar or identical function as a reference polypeptide but does not necessarily comprise a similar or identical amino acid sequence of the reference polypeptide, or possess a similar or identical structure of the reference polypeptide. The reference polypeptide may be a PD-1 polypeptide, a fragment of a PD-1 polypeptide, or an anti-PD-1 antibody. A polypeptide that has a similar amino acid sequence with a reference polypeptide refers to a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the reference polypeptide, which can be a PD-1 polypeptide or an anti-PD-1 antibody as described herein. A polypeptide with similar structure to a reference polypeptide refers to a polypeptide that has a secondary, tertiary, or quaternary structure similar to that of the reference polypeptide, which can be a PD-1 polypeptide or an anti-PD-1 antibody described herein. The structure of a polypeptide can determined by methods known to those skilled in the art, including, but not limited to, X-ray crystallography, nuclear magnetic resonance (NMR), and crystallographic electron microscopy.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

As used herein, the term “variant” when used in relation to a PD-1 polypeptide or to an anti-PD-1 antibody refers to a polypeptide or an anti-PD-1 antibody having one or more amino acid sequence substitutions, deletions, and/or additions as compared to a native or unmodified PD-1 sequence or anti-PD-1 antibody sequence. For example, a PD-1 polypeptide or to an anti-PD-1 antibody refers to a polypeptide or an anti-PD-1 antibody having one or more amino acid sequence substitutions, deletions, and/or additions as compared to a native or unmodified PD-1 sequence or anti-PD-1 antibody sequence can have about 1 to about 25, about 1 to about 20, about 1 to about 15, about 1 to about 10, or about 1 to about 5 amino acid sequence substitutions, deletions, and/or additions as compared to a native or unmodified PD-1 sequence or anti-PD-1 antibody sequence. A PD-1 variant can result from one or more (such as, for example, about 1 to about 25, about 1 to about 20, about 1 to about 15, about 1 to about 10, or about 1 to about 5) changes to an amino acid sequence of a native PD-1. Also by way of example, a variant of an anti-PD-1 antibody can result from one or more (such as, for example, about 1 to about 25, about 1 to about 20, about 1 to about 15, about 1 to about 10, or about 1 to about 5 changes to an amino acid sequence of a native or previously unmodified anti-PD-1 antibody. Variants can be naturally occurring, such as allelic or splice variants, or can be artificially constructed. Polypeptide variants can be prepared from the corresponding nucleic acid molecules encoding the variants.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions can include those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, a variant may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (e.g., an “algorithm”). Methods that may be used to calculate the identity of the aligned nucleic acids or polypeptides include those described in Lesk, A. M., ed., 1988. Computational Molecular Biology, New York: Oxford University Press; Smith, D. W., ed., 1993, Biocomputing Informatics and Genome Projects. New York: Academic Press; Griffin, A. M., et al., 1994, Computer Analysis of Sequence Data, Part I, New Jersey: Humana Press; von Heinje, G., 1987, Sequence Analysis in Molecular Biology, New York: Academic Press; Gribskov, M. et al., 1991, Sequence Analysis Primer, New York: M. Stockton Press; and Carillo et al., 1988, Applied Math., 48:1073.

In calculating percent identity, the sequences being compared can be aligned in a way that gives the largest match between the sequences. An example of a computer program that can be used to determine percent identity is the GCG program package, which includes GAP (Devereux et al., 1984, Nucl. Acid Res., 12:387; Genetics Computer Group, University of Wisconsin, Madison, WI), which is a computer algorithm used to align the two polypeptides or polynucleotides to determine their percent sequence identity. The sequences can be aligned for optimal matching of their respective amino acid or nucleotide sequences (the “matched span” as determined by the algorithm). A gap opening penalty (which is calculated as 3 times the average diagonal, wherein the “average diagonal” is the average of the diagonal of the comparison matrix being used, and the “diagonal” is the score or number assigned to each perfect amino acid match by the particular comparison matrix; and a gap extension penalty (which is usually 1/10 times the gap opening penalty), as well as a comparison matrix such as PAM 250 or BLOSUM 62, are used in conjunction with the algorithm. In some aspects, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. USA 89:10915-10919 for the BLOSUM 62 comparison matrix) is also used by the algorithm. Exemplary parameters for determining percent identity for polypeptides or nucleotide sequences using the GAP program include the following: (i) Algorithm: Needleman et al., 1970, J. Mol. Biol., 48:443-453; (ii) Comparison matrix: BLOSUM 62 from Henikoff et al., Id.; (iii) Gap Penalty: 12 (but with no penalty for end gaps); (iv) Gap Length Penalty: 4; and (v) Threshold of Similarity: 0.

Certain alignment schemes for aligning two amino acid sequences can result in matching only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans a representative number of amino acids, for example, at least 50 contiguous amino acids, of the target polypeptide.

Percent (%) amino acid sequence identity with respect to a reference polypeptide sequence is defined as the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of the practitioner in the art, for instance, using publicly available computer software such as BLAST. BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “derivative” refers to a polypeptide that comprises an amino acid sequence of a reference polypeptide that has been altered by the introduction of amino acid residue substitutions, deletions or additions. The reference polypeptide can be a PD-1 polypeptide or an anti-PD-1 antibody. The term “derivative” as used herein also refers to a PD-1 polypeptide or an anti-PD-1 antibody that has been chemically modified, e.g., by the covalent attachment of any type of molecule to the polypeptide. For example, a PD-1 polypeptide or an anti-PD-1 antibody can be chemically modified, e.g., by glycosylation, acetylation, pegylation, phosphorylation, amidation, derivatization by known protecting/blocking groups, proteolytic cleavage, linkage to a cellular ligand, linkage to a peptide or protein tag molecule, or other protein, etc. The derivatives are modified in a manner that is different from the naturally occurring or starting peptide or polypeptides, either in the type or location of the molecules attached. Derivatives may further include deletion of one or more chemical groups which are naturally present on the peptide or polypeptide. A derivative of a PD-1 polypeptide or an anti-PD-1 antibody may be chemically modified by chemical modifications using techniques known to those of skill in the art, including, but not limited to, specific chemical cleavage, acetylation, formulation, metabolic synthesis by tunicamycin, etc. Further, a derivative of a PD-1 polypeptide or an anti-PD-1 antibody can contain one or more non-classical amino acids. A polypeptide derivative possesses a similar or identical function as the reference polypeptide, which can be a PD-1 polypeptide or an anti-PD-1 antibody described herein, especially an anti-PD-1 monoclonal antibody.

The term “fusion protein” as used herein refers to a polypeptide that includes amino acid sequences of at least two heterologous polypeptides. The term “fusion” when used in relation to a PD-1 polypeptide or to an anti-PD-1 antibody refers to the joining, fusing, or coupling of a PD-1 polypeptide or an anti-PD-1 antibody, variant and/or derivative thereof, with a heterologous peptide or polypeptide. In some aspects, the fusion protein retains the biological activity of the PD-1 polypeptide or the anti-PD-1 antibody. In some aspects, the fusion protein includes a PD-1 antibody VH region, VL region, VH CDR (one, two or three VH CDRs), and/or VL CDR (one, two or three VL CDRs) coupled, fused, or joined to a heterologous peptide or polypeptide, wherein the fusion protein binds to an epitope on a PD-1 protein or peptide. Fusion proteins may be prepared via chemical coupling reactions as practiced in the art, or via molecular recombinant technology.

As used herein, the term “composition” refers to a product containing specified component ingredients (e.g., a polypeptide or an antibody provided herein) in, optionally, specified or effective amounts, as well as any desired product which results, directly or indirectly, from the combination or interaction of the specific component ingredients in, optionally, the specified or effective amounts.

As used herein, the term “carrier” includes pharmaceutically acceptable carriers, excipients, diluents, vehicles, or stabilizers that are nontoxic to the cell or mammal being exposed thereto at the dosages and concentrations employed. Often, the physiologically acceptable carrier is an aqueous pH buffered solution. Examples of physiologically acceptable carriers include buffers such as phosphate, citrate, succinate, and other organic acids; antioxidants including ascorbic acid; low molecular weight (e.g., less than about 10 amino acid residues) polypeptides; proteins, such as serum albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, sucrose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant, complete or incomplete), excipient, or vehicle with which the therapeutic is administered. Such carriers, including pharmaceutical carriers, can be sterile liquids, such as water and oils, including those of petroleum, animal, vegetable or synthetic origin, such as peanut oil, soy bean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, (1990) Mack Publishing Co., Easton, PA. Compositions, including pharmaceutical compounds, can contain a therapeutically effective amount of an anti-PD-1 antibody in isolated or purified form, together with a suitable amount of carrier so as to provide the form for proper administration to the subject (e.g., patient). The composition or formulation should suit the mode of administration.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference. Remington's Pharmaceutical Sciences, (1990) Mack Publishing Co., Easton, PA, which is hereby incorporated by reference in its entirety.

As used herein, the term “pharmaceutically acceptable” or “pharmacologically acceptable” refers to molecular entities, formulations and compositions that do not produce an adverse, allergic, or other untoward or unwanted reaction when administered, as appropriate, to an animal, such as a human. The preparation of a pharmaceutical composition comprising an antibody or additional active ingredient are known to those of skill in the art in light of the present disclosure, as exemplified by Remington's Pharmaceutical Sciences, Id. Moreover, for animal (e.g., human) administration, it will be understood that preparations should meet sterility, pyrogenicity, general safety, and purity standards as required by a regulatory agency of the Federal or a state government, such as the FDA Office of Biological Standards or as listed in the U.S. Pharmacopeia, European Pharmacopeia. or other generally recognized Pharmacopeia for use in animals, and more particularly, in humans.

The term “pharmaceutical formulation” refers to a preparation which is in such form as to permit the biological activity of the active ingredient (e.g., an isolated antibody as described herein, including, but not limited to an anti-PD-1 antibody) to be effective, and which contains no additional components that would be are unacceptably toxic to a subject to whom the formulation would be administered. Such a formulation can be sterile, i.e., aseptic or free from all living microorganisms and their spores, etc.

The term “package insert” is used to refer to instructions customarily included in commercial packages of therapeutic products, that contain information about the indications, usage, dosage, administration, contraindications and/or warnings concerning the use of such therapeutic products.

The terms “antibody,” “immunoglobulin,” and “Ig” are used interchangeably herein in a broad sense and specifically cover, for example, individual anti-PD-1 antibodies, such as the monoclonal antibodies described herein, (including agonist, antagonist, neutralizing antibodies, full length or intact monoclonal antibodies, peptide fragments of antibodies that maintain antigen binding activity); anti-PD-1 antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-PD-1 antibodies, and fragments of anti-PD-1, as described herein. An antibody can be human, humanized, chimeric and/or affinity matured. An antibody may be from other species, for example, mouse, rat, rabbit, etc. The term “antibody” is intended to include a polypeptide product of B cells within the immunoglobulin class of polypeptides that is able to bind to a specific molecular antigen. An antibody is typically composed of two identical pairs of polypeptide chains, wherein each pair has one heavy chain (about 50-70 kDa) and one light chain (about 25 kDa); and wherein the amino-terminal portion of the heavy and light chains includes a variable region of about 100 to about 130 or more amino acids and the carboxy-terminal portion of each chain includes a constant region (See, Borrebaeck (ed.), 1995, Antibody Engineering, Second Ed., Oxford University Press.; Kuby, 1997 Immunology, Third Ed., W. H. Freeman and Company, New York). In some aspects, the specific molecular antigen bound by an antibody provided herein includes a PD-1 polypeptide, a PD-1 peptide fragment, or a PD-1 epitope. An antibody or a peptide fragment thereof that binds to a PD-1 antigen can be identified, for example, by immunoassays, BIAcore, or other techniques known to those of skill in the art. An antibody or a fragment thereof binds specifically to a PD-1 antigen when it binds to a PD-1 antigen with higher affinity than to any cross-reactive antigen as determined using experimental techniques, such as radioimmunoassays (RIA) and enzyme linked immunosorbent assays (ELISAs). Typically, a specific or selective binding reaction will be at least twice background signal or noise, and more typically more than 5-10 times background signal or noise. See, e.g., Paul, ed., 1989, Fundamental Immunology Second Edition, Raven Press, New York at pages 332-336 for a discussion regarding antibody specificity.

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments (e.g., antigen-binding fragments such as PD-1 binding fragments) of any of the above. A binding fragment refers to a portion of an antibody heavy or light chain polypeptide, such as a peptide portion, that retains some or all of the binding activity of the antibody from which the fragment is derived. Non-limiting examples of functional fragments (e.g., antigen-binding fragments such as PD-1 binding fragments) include single-chain Fvs (scFv) (e.g., including monospecific, bispecific, etc.), Fab fragments, F(ab′) fragments. F(ab)₂ fragments, F(ab′)₂ fragments, disulfide-linked Fvs (sdFv), Fd fragments. Fv fragments, diabodies, triabodies, tetrabodies and minibodies. In particular, antibodies provided herein include immunoglobulin molecules and immunologically active portions of immunoglobulin molecules, for example, antigen binding domains or molecules that contain an antigen-binding site that binds to a PD-1 antigen, (e.g., one or more complementarity determining regions (CDRs) of an anti-PD-1 antibody). Description of such antibody fragments can be found in, for example, Harlow and Lane, 1989, Antibodies: A Laboratory Manual, Cold Spring Harbor Laboratory. New York; Myers (ed.), Molec. Biology and Biotechnology: A Comprehensive Desk Reference, New York: VCH Publisher, Inc; Huston et al., 1993, Cell Biophysics, 22:189-224; Pluckthun and Skerra, 1989, Meth. Enzymol., 178:497-515 and in Day, E. D., 1990, Advanced Immunochemistry. Second Ed., Wiley-Liss, Inc., New York, NY. The antibodies provided herein can be of any type (e.g., IgG, IgE. IgM, IgD, IgA and IgY), any class (e.g., IgG1, IgG2, IgG3, IgG4, IgA1 and IgA2), or any subclass (e.g., IgG2a and IgG2b) of immunoglobulin molecule. Anti-PD-1 antibodies can be agonistic antibodies or antagonistic antibodies. In some aspects, the anti-PD-1 antibodies can be fully human, such as fully human monoclonal anti-PD-1 antibodies. In some aspects, the anti-PD-1 antibodies can be humanized, such as humanized monoclonal anti-PD-1 antibodies. In some aspects, the antibodies provided herein can be IgG antibodies, or a class (e.g., human IgG1 or IgG4) or subclass thereof, in particular, IgG1 subclass antibodies.

A four-chain antibody unit is a heterotetrameric glycoprotein composed of two identical light (L) chains and two identical heavy (H) chains. In the case of IgGs, the molecular weight of the four-chain (unreduced) antibody unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. At the N-terminus, each H chain has a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its carboxy terminus. The V_{L} domain is aligned with the V_{H} domain, and the C_{L} domain is aligned with the first constant domain of the heavy chain (C_{H1}). Particular amino acid residues are believed to form an interface between the light chain and heavy chain variable domains. The pairing of a V_{H} and V_{L} together forms a single antigen-binding site, although certain V_{H} and V_{L} domains can bind antigen without pairing with a V_{L} or V_{H} domain, respectively. The basic structure of immunoglobulin molecules is understood by those having skill in the art. For example, the structure and properties of the different classes of antibodies may be found in Terr, Abba I. et al., 1994, Basic and Clinical Immunology, 8th edition, Appleton & Lange. Norwalk, CT, page 71 and Chapter 6.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

A “fragment antigen-binding fragment (Fab)” is a region of an antibody that binds to antigen. A Fab comprises constant and variable regions from both heavy and light chains.

As used herein, the term “antigen” or “target antigen” is a predetermined molecule to which an antibody can selectively bind. A target antigen can be a polypeptide, peptide, carbohydrate, nucleic acid, lipid, hapten, or other naturally occurring or synthetic compound. In some aspects, a target antigen can be a small molecule. In some aspects, the target antigen can be a polypeptide or peptide, e.g., PD-1.

As used herein, the term “antigen binding fragment.” “antigen binding domain.” “antigen binding region,” and similar terms refer to that portion of an antibody which includes the amino acid residues that interact with an antigen and confer on the antibody as binding agent its specificity and affinity for the antigen (e.g., the CDRs of an antibody are antigen binding regions). The antigen binding region can be derived from any animal species, such as rodents (e.g., rabbit, rat, or hamster) and humans. In some aspects, the antigen binding region can be of human origin.

An “isolated” antibody is substantially free of cellular material or other contaminating proteins from the cell or tissue source and/or other contaminant components from which the antibody is derived, or is substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of an antibody that have less than about 30%, 25%, 20%, 15%, 10%, 5%, or 1% (by dry weight) of heterologous protein (also referred to herein as a “contaminating protein”). In some aspects, when the antibody is recombinantly produced, it is substantially free of culture medium, e.g., culture medium represents less than about 20%, 15%, 10%, 5%, or 1% of the volume of the protein preparation. In some aspects, when the antibody is produced by chemical synthesis, it is substantially free of chemical precursors or other chemicals, for example, it is separated from chemical precursors or other chemicals which are involved in the synthesis of the protein. Accordingly such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and can include enzymes, hormones, and other proteinaceous or nonproteinaceous solutes. In some aspects, the antibody is purified (1) to greater than or equal to 95% by weight of the antibody, as determined by the Lowry method (Lowry et al., 1951, J. Bio. Chem., 193:265-275), such as 95%, 96%, 97%, 98%, or 99%, by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody also includes the antibody in situ within recombinant cells since at least one component of the antibody's natural environment will not be present. An isolated antibody is typically prepared by at least one purification step. In some aspects, the antibodies provided herein are isolated.

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256:495-497, 1975), or by recombinant DNA methods.

As used herein, the term “binds” or “binding” refers to an interaction between molecules including, for example, to form a complex. Illustratively, such interactions embrace non-covalent interactions, including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site of an antibody and its epitope on a target (antigen) molecule, such as PD-1, is the affinity of the antibody or functional fragment for that epitope. The ratio of association (kₒₙ) to dissociation (k_{off}) of an antibody to a monovalent antigen (kₒₙ/k_{off}) is the association constant Kₐ, which is a measure of affinity. The value of K varies for different complexes of antibody and antigen and depends on both kₒₙ and k_{off}. The association constant Kₐ for an antibody provided herein may be determined using any method provided herein or any other method known to those skilled in the art. The affinity at one binding site does not always reflect the true strength of the interaction between an antibody and an antigen. When complex antigens containing multiple, repeating antigenic determinants come into contact with antibodies containing multiple binding sites, the interaction of antibody with antigen at one site will increase the probability of an interaction at a second binding site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{d}). Affinity can be measured by common methods known in the art, including those described herein. Low-affinity antibodies generally bind antigen slowly and tend to dissociate readily, while high-affinity antibodies generally bind antigen faster and tend to remain bound longer to antigen. A variety of methods for measuring binding affinity are known in the art, any of which may be used for purposes of the present disclosure. Specific illustrative aspects include the following: In some aspects, the “K_{d}” or “K_{d} value” is measured by assays known in the art, for example, by a binding assay. The K_{d} can be measured in a radiolabeled antigen binding assay (RIA), for example, performed with the Fab portion of an antibody of interest and its antigen (Chen, et al., 1999, J. Mol. Biol., 293:865-881). The K_{d} or K_{d} value may also be measured by using surface plasmon resonance (SPR) assays (by BIAcore) using, for example, a BIAcore™-2000 or a BIAcore™-3000 (BIAcore, Inc., Piscataway, NJ), or by biolayer interferometry (BLI) using, for example, the OctetQK384 system (ForteBio, Menlo Park, CA), or by quartz crystal microbalance (QCM) technology. An “on-rate” or “rate of association” or “association rate” or “kₒₙ” can also be determined with the same surface plasmon resonance or biolayer interferometry techniques described above, using, for example, a BIAcore™-2000 or a BIAcore™-3000 (BIAcore, Inc., Piscataway, NJ), or the OctetQK384 system (ForteBio. Menlo Park, CA).

Disclosed herein are isolated antibodies including, but not limited to, anti-PD-1 antibodies, antibodies that specifically bind to PD-1, antibodies that are specific for PD-1, antibodies that specifically bind to a PD-1 epitope, antibodies that selectively bind to a PD-1 epitope, and antibodies that preferentially binds to PD-1. The terms “anti-PD-1 antibody,” “anti-PD-1 monoclonal antibody”, “monoclonal PD-1”, “an antibody that specifically binds to PD-1,” or “antibody that is specific for PD-1,” “antibodies that specifically bind to a PD-1 epitope,” “an antibody that selectively binds to PD-1,” “antibodies that selectively bind to a PD-1 epitope,” “an antibody that preferentially binds to PD-1”, and analogous terms refer to antibodies capable of binding PD-1, i.e., WT PD-1, with sufficient affinity and specificity, particularly compared with mutants of PD-1.

By “specifically binds” is meant that an antibody recognizes and physically interacts with its cognate antigen (for example PD-1) and does not significantly recognize and interact with other antigens; such an antibody may be a polyclonal antibody or a monoclonal antibody, which are generated by techniques that are well known in the art.

“Preferential binding” of the anti-PD-1 antibodies as provided herein may be determined or defined based on the quantification of fluorescence intensity of the antibodies” binding to PD-1, i.e., PD-1 polypeptide, or PD-1 WT, or PD-1 expressed on cells versus an appropriate control, such as binding to variant PD-1, or to cells expressing a variant form of PD-1, for example, molecularly engineered cells, cell lines or tumor cell isolates. Preferential binding of an anti-PD-1 antibody as described to a PD-1 WT-expressing cell is indicated by a measured fluorescent binding intensity (MFI) value, as assessed by cell flow cytometry, of at least 2-fold, at least 3-fold, at least 4-fold, at least 5-fold, at least 6-fold, at least 7-fold, at least 8-fold, at least 10-fold, at least 15-fold, at least 20-fold or greater, as compared with binding of the antibody to a mutant PD-1 polypeptide or a mutant PD-1-expressing cell, wherein the antibody to be assayed is directly or indirectly detectable by a fluorescent label or marker, such as FITC. In some aspects, the antibody to be assayed is directly labeled with a fluorescent marker, such as FITC. In some aspects, an anti-PD-1 antibody that preferentially or selectively binds PD-1 exhibits an MFI value of from 1.5-fold to 25-fold, or from 2-fold to 20-fold, or from 3-fold to 15-fold, or from 4-fold to 8-fold, or from 2-fold to 10-fold, or from 2-fold to 5-fold or more greater than the MFI value of the same antibody for binding a PD-1 or a PD-1 variant. Fold-fluorescence intensity values between and equal to all of the foregoing are intended to be included. In some aspects, the anti-PD-1 antibodies specifically and preferentially bind to a PD-1 polypeptide, such as a PD-1 antigen, peptide fragment, or epitope (e.g., human PD-1 such as a human PD-1 polypeptide, antigen or epitope). An antibody that specifically binds to PD-1, (e.g., wild type human PD-1) can bind to the extracellular domain (ECD) or a peptide derived from the ECD of PD-1. An antibody that specifically binds to a PD-1 antigen (e.g., human PD-1) can be cross-reactive with related antigens (e.g., cynomolgus (cyno) PD-1). In some aspects, an antibody that specifically binds to a PD-1 antigen does not cross-react with other antigens. An antibody that specifically binds to a PD-1 antigen can be identified, for example, by immunofluorescence binding assays, immunohistochemistry assay methods, immunoassay methods, Biacore, or other techniques known to those of skill in the art.

In some aspects, an antibody that binds to PD-1, as described herein, has a equilibrium dissociation constant (K_{D}) of less than or equal to 50 nM, 40 nM, 30 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 0.9 nM, 0.8 nM, 0.7 nM, 0.6 nM, 0.5 nM, 0.4 nM, 0.3 nM, 0.2 nM, 0.1 nM, 0.01 mM, or 0.001 nM and/or is greater than or equal to 0.001 nM. K_{D} is the dissociation constant and is the concentration of ligand, wherein half of the ligand binding sites on the protein are occupied in the system equilibrium. The K_{D} is calculated by dividing the k_{off} value by the kₒₙ value. It is also equal to the product of the concentrations of the ligand and protein divided by the concentration of the protein ligand complex once equilibrium is reached. In some aspects, an anti-PD-1 antibody binds to an epitope of PD-1 that is conserved among PD-1 proteins from different species (e.g., between human and mouse PD-1). An antibody binds specifically to a PD-1 antigen when it binds to a PD-1 antigen with higher affinity than to any cross reactive antigen as determined using experimental techniques, such as radioimmunoassays (RIA) and enzyme linked immunosorbent assays (ELISAs). Typically a specific or selective reaction will be at least twice background signal or noise and can be more than 10 times background. See, e.g., Paul, ed., 1989, Fundamental Immunology Second Edition, Raven Press, New York at pages 332-336 for a discussion regarding antibody specificity. In some aspects, the extent of binding of the antibody to a “non-target” protein will be less than about 10% of the binding of the antibody to its particular target protein, for example, as determined by fluorescence activated cell sorting (FACS) analysis or radioimmunoprecipitation (RIA).

As used herein, in reference to an antibody, the term “heavy (H) chain” refers to a polypeptide chain of about 50-70 kDa, wherein the amino-terminal portion includes a variable (V) region (also called V domain) of about 115 to 130 or more amino acids and a carboxy-terminal portion that includes a constant (C) region. The constant region (or constant domain) can be one of five distinct types. (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes (e.g., isotypes) of antibodies, namely, IgA, IgD, IgE, IgG and IgM, respectively, including four subclasses of IgG, namely IgG1, IgG2, IgG3 and IgG4. An antibody heavy chain can be a human antibody heavy chain.

As used herein in reference to an antibody, the term “light (L) chain” refers to a polypeptide chain of about 25 kDa, wherein the amino-terminal portion includes a variable domain of about 100 to about 110 or more amino acids and a carboxy-terminal portion that includes a constant region. The approximate length of a light chain (both the V and C domains) is 211 to 217 amino acids. There are two distinct types of light chains, referred to as kappa (κ) and lambda (λ), based on the amino acid sequence of the constant domains. Light chain amino acid sequences are well known in the art. An antibody light chain can be a human antibody light chain.

As used herein, the term “variable (V) region” or “variable (V) domain” refers to a portion of the light (L) or heavy (H) chains of an antibody polypeptide that is generally located at the amino-terminus of the L or H chain. The H chain V domain has a length of about 115 to 130 amino acids, while the L chain V domain is about 100 to 110 amino acids in length. The H and L chain V domains are used in the binding and specificity of each particular antibody for its particular antigen. The V domain of the H chain can be referred to as “VH.” The V region of the L chain can be referred to as “VL.” The term “variable” refers to the fact that certain segments of the V domains differ extensively in sequence among different antibodies. While the V domain mediates antigen binding and defines specificity of a particular antibody for its particular antigen, the variability is not evenly distributed across the 110-amino acid span of antibody V domains. Instead, the V domains consist of less variable (e.g., relatively invariant) stretches called framework regions (FRs) of about 15-30 amino acids separated by shorter regions of greater variability (e.g., extreme variability) called “hypervariable regions” or “complementarity determining regions” (CDRs) that are each about 9-12 amino acids long or 3-17 amino acids long. The V domains of antibody H and L chains each comprise four FRs, largely adopting a β sheet configuration, connected by three hypervariable regions, called, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., 1991, Sequences of Proteins of Immunological Interest, 5th Ed. Public Health Service, National Institutes of Health. Bethesda, MD). The C domains are not involved directly in binding an antibody to an antigen, but exhibit various effector functions, such as antibody dependent cellular cytotoxicity (ADCC) and complement dependent cytotoxicity (CDC). The V domains differ extensively in sequence among different antibody classes or types. The variability in sequence is concentrated in the CDRs, which are primarily responsible for the interaction of the antibody with antigen. In some aspects, the variable domain of an antibody is a human or humanized variable domain.

As used herein, the terms “complementarity determining region,” “CDR,” “hypervariable region,” “HVR,” and “HV” are used interchangeably. A “CDR” or “complementarity determining region” is a region of hypervariability interspersed within regions that are more conserved, termed “framework regions” (FR). A “CDR” refers to one of three hypervariable regions (H1, H2 or H3) within the non-framework region of the antibody VH β-sheet framework, or to one of three hypervariable regions (L1, L2 or L3) within the non-framework region of the antibody VL β-sheet framework. The term, when used herein, refers to the regions of an antibody V domain that are hypervariable in sequence and/or form structurally defined loops. Generally, antibodies comprise six hypervariable regions: three (H1, H2, H3) in the VH domain and three (L1, L2, L3) in the VL domain. Accordingly, CDRs are typically highly variable sequences interspersed within the framework region sequences of the V domain. “Framework” or “FR” residues are those variable region residues flanking the CDRs. FR residues are present, for example, in chimeric, humanized, human, domain antibodies, diabodies, linear antibodies, and bispecific antibodies.

A number of hypervariable region delineations are in use and are encompassed herein. CDR regions are well known to those skilled in the art and have been defined by, for example, Kabat as the regions of most hypervariability within the antibody V domains (Kabat et al., 1977, J. Biol. Chem., 252:6609-6616; Kabat, 1978, Adv. Prot. Chem., 32:1-75). The Kabat CDRs are based on sequence variability and are the most commonly used (see, e.g., Kabat et al., 1991, Sequences of Proteins of Immunological Interest, 5th Ed. Public Health Service, National Institutes of Health, Bethesda, MD). CDR region sequences also have been defined structurally by Chothia as those residues that are not part of the conserved β-sheet framework, and thus are able to adopt different conformations (Chothia et al., 1987, J. Mol. Biol., 196:901-917). Chothia refers instead to the location of the structural loops. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both numbering systems and terminologies are well recognized in the art.

Recently, a universal numbering system has been developed and widely adopted. ImMunoGeneTics (IMGT) Information System® (Lafranc et al., 2003, Dev. Comp. Immunol., 27(1); 55-77). IMGT is an integrated information system specializing in immunoglobulins (Ig), T cell receptors (TR) and the major histocompatibility complex (MHC) of human and other vertebrates. Herein, the CDRs are referred to in terms of both the amino acid sequence and the location within the light or heavy chain. As the “location” of the CDRs within the structure of the immunoglobulin V domain is conserved between species and present in structures called loops, by using numbering systems that align variable domain sequences according to structural features, CDR and framework residues and are readily identified. This information can be used in grafting and in the replacement of CDR residues from immunoglobulins of one species into an acceptor framework from, typically, a human antibody. An additional numbering system (AHon) has been developed by Honegger et al., 2001, J. Mol. Biol., 309:657-670. Correspondence between the numbering system, including, for example, the Kabat numbering and the IMGT unique numbering system, is well known to one skilled in the art (see, e.g., Kabat, Id; Chothia et al., Id.; Martin, 2010, Antibody Engineering, Vol. 2, Chapter 3, Springer Verlag; and Lefranc et al., 1999, Nuc. Acids Res., 27:209-212).

CDR region sequences have also been defined by AbM, Contact and IMGT. The AbM hypervariable regions represent a compromise between the Kabat CDRs and Chothia structural loops, and are used by Oxford Molecular's AbM antibody modeling software (see, e.g., Martin, 2010, Antibody Engineering, Vol. 2, Chapter 3, Springer Verlag). The “contact” hypervariable regions are based on an analysis of the available complex crystal structures. The residues from each of these hypervariable regions or CDRs are noted below.

Exemplary delineations of CDR region sequences are illustrated in Table 2. The positions of CDRs within a canonical antibody variable region have been determined by comparison of numerous structures (Al-Lazikani et al., 1997, J. Mol. Biol., 273:927-948); Morea et al., 2000, Methods, 20:267-279). Because the number of residues within a hypervariable region varies in different antibodies, additional residues relative to the canonical positions are conventionally numbered with a, b, c and so forth next to the residue number in the canonical variable region numbering scheme (Al-Lazikani et al., Id). Such nomenclature is similarly well known to those skilled in the art.

TABLE 2
Exemplary Delineations of CDR Region Sequences

| | IMGT | Kabat |
|---|---|---|
| V_{H} CDR1 | 28-35 | 31-35 |
| V_{H} CDR2 | 51-58 | 47-56 |
| V_{H} CDR3 | 97-103 | 99-103 |
| V_{L} CDR1 | 27-32 | 24-34 |
| V_{L} CDR2 | 50-52 | 50-56 |
| V_{L} CDR3 | 89-97 | 89-97 |

An “affinity matured” antibody is one with one or more alterations (e.g., amino acid sequence variations, including changes, additions and/or deletions) in one or more HVRs thereof that result in an improvement in the affinity of the antibody for antigen, compared to a parent antibody which does not possess those alteration(s). In some aspects, affinity matured antibodies will have nanomolar or even picomolar affinities for the target antigen, such as PD-1. Affinity matured antibodies are produced by procedures known in the art. For reviews, see Hudson and Souriau, 2003, Nature Medicine, 9:129-134; Hoogenboom, 2005, Nature Biotechnol., 23:1105-1116; Quiroz and Sinclair, 2010, Revista Ingeneria Biomedia, 4:39-51.

A “chimeric” antibody is one in which a portion of the H and/or L chain, e.g., the V domain, is identical with or homologous to a corresponding amino acid sequence in an antibody derived from a particular species or belonging to a particular antibody class or subclass, while the remainder of the chain(s), e.g., the C domain, is identical with or homologous to corresponding sequences in antibodies derived from another species or belonging to another antibody class or subclass, as well as a fragment of such an antibody, so long as it exhibits the desired biological activity (see, e.g., U.S. Pat. No. 4,816,567; and Morrison et al., 1984, Proc. Natl. Acad. Sci. USA, 81:6851-6855).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter. European Patent No. 0,239,400 B1; Padlan, E. A. et al., European Patent Application No. 0,519,596 A1; Queen et al. (1989) Proc. Natl. Acad. Sci. USA. Vol 86:10029-10033). The terms “human antibody” and “fully human antibody” are used interchangeably herein and refer to an antibody that possesses an amino acid sequence which corresponds to that of an antibody produced by a human and/or has been made using any of the techniques for making human antibodies as practiced by those skilled in the art. This definition of a human antibody specifically excludes a humanized antibody comprising non-human antigen-binding residues. Human antibodies can be produced using various techniques known in the art, including phage-display libraries (Hoogenboom et al., 1991, J. Mol. Biol., 227:381; Marks et al., 1991. J. Mol. Biol., 222:581 and yeast display libraries (Chao et al., 2006. Nature Protocols, 1:755-768). Also available for the preparation of human monoclonal antibodies are methods described in Cole et al., 1985 Monoclonal Antibodies and Cancer Therapy, Alan R. Liss, p. 77; Boerner et al., 1991, J. Immunol., 147(1); 86-95. See also van Dijk et al., 2001, Curr. Opin. Pharmacol., 5:368-74. Human antibodies can be prepared by administering an antigen to a transgenic animal whose endogenous Ig loci have been disabled, e.g., a mouse, and that has been genetically modified to harbor human immunoglobulin genes which encode human antibodies, such that human antibodies are generated in response to antigenic challenge (see, e.g., Jakobovits, A., 1995, Curr. Opin. Biotechnol. 6(5); 561-566; Brüggemann et al., 1997 Curr. Opin. Biotechnol., 8(4); 455-8; and U.S. Pat. Nos. 6,075,181 and 6,150,584 regarding XENOMOUSE™ technology). See also, for example, Li et al., 2006, Proc. Natl. Acad. Sci. USA, 103:3557-3562 regarding human antibodies generated via a human B-cell hybridoma technology. In some aspects, human antibodies comprise a variable region and constant region of human origin. “Fully human” anti-PD-1 antibodies, in some aspects, can also encompass antibodies which bind PD-1 polypeptides and are encoded by nucleic acid sequences which are naturally occurring somatic variants of human germline immunoglobulin nucleic acid sequence. In some aspects, the anti-PD-1 antibodies provided herein are fully human antibodies. The term “fully human antibody” includes antibodies having variable and constant regions corresponding to human germline immunoglobulin sequences as described by Kabat et al. (See Kabat et al., 1991, Sequences of Proteins of Immunological Interest, Fifth Edition, U.S. Department of Health and Human Services, NIH Publication No. 91-3242). The phrase “recombinant human antibody” includes human antibodies that are prepared, expressed, created, or isolated by recombinant means, such as antibodies expressed using a recombinant expression vector transfected into a host cell; antibodies isolated from a recombinant, combinatorial human antibody library; antibodies isolated from an animal (e.g., a mouse or cow) that is transgenic and/or transchromosomal for human immunoglobulin genes (see e.g., Taylor, L. D. et al., 1992, Nucl. Acids Res. 20:6287-6295); or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat et al., 1991. Sequences of Proteins of Immunological Interest, Fifth Edition, U.S. Department of Health and Human Services, NIH Publication No. 91-3242). In some aspects, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

As used herein, the term “epitope” is the site(s) or region(s) on the surface of an antigen molecule to which a single antibody molecule binds, such as a localized region on the surface of an antigen, e.g., a PD-1 polypeptide that is capable of being bound by one or more antigen binding regions of an anti-PD-1 antibody. An epitope can be immunogenic and capable of eliciting an immune response in an animal. Epitopes need not necessarily be immunogenic. Epitopes often consist of chemically active surface groupings of molecules such as amino acids or sugar side chains and have specific three dimensional structural characteristics as well as specific charge characteristics. An epitope can be a linear epitope and a conformational epitope. A region of a polypeptide contributing to an epitope can be contiguous amino acids of the polypeptide, forming a linear epitope, or the epitope can be formed from two or more non-contiguous amino acids or regions of the polypeptide, typically called a conformational epitope. The epitope may or may not be a three-dimensional surface feature of the antigen. In some aspects, a PD-1 epitope is a three-dimensional surface feature of a PD-1 polypeptide. In some aspects, a PD-1 epitope is linear feature of a PD-1 polypeptide.

An antibody binds “an epitope” or “essentially the same epitope” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies to antigen is measured using a detectable signal, e.g., radioactive, fluorescent or enzyme labels.

The term “compete” when used in the context of anti-PD-1 antibodies that compete for the same epitope or binding site on a PD-1 target protein or peptide thereof means competition as determined by an assay in which the antibody under study, or binding fragment thereof, prevents, blocks, or inhibits the specific binding of a reference molecule (e.g., a reference ligand, or reference antigen binding protein, such as a reference antibody) to a common antigen (e.g., PD-1 or a fragment thereof). Numerous types of competitive binding assays can be used to determine if a test antibody competes with a reference antibody for binding to PD-1 (e.g., human PD-1). Examples of assays that can be employed include solid phase direct or indirect radioimmunoassay (RIA); solid phase direct or indirect enzyme immunoassay (EIA); sandwich competition assay (see, e.g., Stahli et al., 1983, Methods in Enzymology 9:242-253); solid phase direct biotin-avidin EIA (see, e.g., Kirkland et al., 1986. J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using labeled iodine (1125 label) (see. e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of a purified antigen (e.g., PD-1) bound to a solid surface, or cells bearing either of an unlabeled test antigen binding protein (e.g., test anti-PD-1 antibody) or a labeled reference antigen binding protein (e.g., reference anti-PD-1 antibody). Competitive inhibition can be measured by determining the amount of label bound to the solid surface or cells in the presence of a known amount of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference antibody causing steric hindrance to occur. Additional details regarding methods for determining competitive binding are described herein. Usually, when a competing antibody protein is present in excess, it will inhibit specific binding of a reference antibody to a common antigen by at least 15%, or at least 20%, for example, without limitation, 25%, 30%, 35%, 40%, 45%, 50%, 55%, 60%, 65%, 70% or 75% or greater, as well as percent amounts between the amounts stated. In some aspects, binding can be inhibited by at least 80%, 85%, 90%, 95%, 96% or 97%, 98%, 99% or more.

As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-PD-1 antibody can prevent, inhibit, block, or reduce the binding interaction between PD-1 and PD-L1, thus preventing, blocking, inhibiting, or reducing the immune system functions associated with the PD-1/PD-L1 interaction. The terms block, inhibit, and neutralize are used interchangeably herein and refer to the ability of the anti-PD-1 antibodies to prevent or otherwise disrupt or reduce the PD-1/PD-L1 interaction.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

PD-1 immune checkpoint has been investigated in pathogenesis and treatments for cancer and autoimmune diseases. Cells that express PD-1 (PD-1⁺ cells) draw ever-increasing attention in cancer and autoimmune disease research although the role of PD-1⁺ cells in the progression and treatments of these diseases remains largely ambiguous. One definite approach to elucidate their roles is to deplete these cells in disease settings and examine how the depletion impacts disease progression and treatments. To execute the depletion, a depleting antibody (D-αPD-1) that specifically ablates PD-1⁺ cells was designed and generated. D-αPD-1 has the same variable domains as an anti-mouse PD-1 blocking antibody (RMP1-14). The constant domains of D-αPD-1 were derived from mouse IgG2a heavy and κ-light chain, respectively. D-αPD-1 was verified to bind with mouse PD-1 as well as mouse FcγRIV, an immuno-activating Fc receptor. The cell depletion effect of D-αPD-1 was confirmed in vivo using a PD-1′ cell transferring model. Since transferred PD-1′ cells, EL4 cells, are tumorigenic and EL4 tumors are lethal to host mice, the depleting effect of D-αPD-1 was also manifested by an absolute survival among the antibody-treated mice while groups receiving control treatments had median survival time of merely approximately 30 days. Furthermore, it was also found that D-αPD-1 leads to elimination of PD-1⁺ cells through antibody-dependent cell-mediate phagocytosis (ADCP) and complement-dependent cytotoxicity (CDC) mechanisms. These results, altogether, confirmed the specificity and effectiveness of D-αPD-1. The results also highlighted that D-αPD-1 can be used to study PD-1+ cells in cancer and autoimmune diseases and as a therapeutic for these diseases.

PD-1 immune checkpoint is an important mechanism to maintain immunostasis. The checkpoint counteracts immune stimulatory signals and hence, negatively regulates immune responses. PD-1 immune checkpoint is triggered when PD-1, a receptor, engages with its ligands, PD-L1 or PD-L2. PD-1 was first found to be expressed on activated B and T cells when they differentiate into effector cells (Francisco, L. M., et al., Immunol Rev, 2010. 236: p. 219-42; Agata. Y., et al., Int Immunol. 1996. 8(5); p. 765-72; and Yamazaki. T. et al., J Immunol, 2002. 169(10); p. 5538-45). Recently. PD-1 was also found to be expressed on other immune cells including activated NK cells, macrophages, and innate lymphoid cells (Patsoukis, N., et al., Revisiting the PD-1 pathway. Sci Adv, 2020. 6(38); and Okazaki. T., et al., Nature Immunology, 2013. 14(12); p. 1212-1218). PD-L1 and PD-L2 are found to express on a wide range of cells including dendritic cells, macrophages, B cells, and some non-immune peripheral cells (Zhai, Y., R. et al., Front Immunol, 2021. 12; p. 645699). To date, the PD-1 immune checkpoint has been connected with pathogenesis and treatments for several types of disorders including autoimmune diseases, cancer, and chronic infections (Joller, N., et al., Immunol Rev, 2012. 248(1); p. 122-39; Salama, A. D., et al., J Exp Med, 2003. 198(1); p. 71-8; Okazaki, T., et al., Nat Immunol, 2013. 14(12); p. 1212-81 and Fife, B. T., et al., Nat Immunol, 2009. 10(11); p. 1185-92). For example, the knockout of PD-1 in NOD mice exacerbates type-1 diabetes in mice; the knockout in C57B/L6 mice confer a lupus-like phenotype in these mice (Nishimura, H., et al., Immunity, 1999. 11(2); p. 141-51). The broad implication of PD-1 immune checkpoint in various diseases also brought attention to cells that express PD-1, generally termed as PD-1-positive (PD-1⁺) cells. PD-1⁺ cells encompass different populations of cells and play drastically different roles in different disease settings.

In the context of autoimmune disease, PD-1⁺ cells refer to effector T and B cells that exert autoimmune attacks although PD-1⁺ Tregs may also influence the initiation and progression of certain diseases. Tissue infiltration of PD-1⁺ cells was found to increase the progression of autoimmune diseases (Salama, A. D., et al., J Exp Med, 2003. 198(1); p. 71-8). Another fact is that PD-1⁺ cell proliferation worsens the condition of mice and patients with autoimmune disease (Salama, A. D., et al., J Exp Med, 2003. 198(1); p. 71-8; Godwin, J. L., et al., J Immunother Cancer, 2017. 5: p. 40; Ansari, M. J., et al., J Exp Med, 2003. 198(1); p. 63-9; and Hughes, J., et al., Diabetes Care, 2015. 38(4); p, e55-7). Interestingly, the PD-1 immune checkpoint intrinsically functions to suppress autoimmunity. The observation that autoimmune diseases start and progress despite the normality of the PD-1 immune checkpoint suggests the immune checkpoint may be overridden by other autoimmunity-driving factors (Joller. N., et al., Immunol Rev, 2012. 248(1); p. 122-3; and Okazaki. T., et al., Nat Immunol. 2013. 14(12); p. 1212-8). Clinically and preclinically, it has been reported that a blockade of the PD-1 immune checkpoint, a treatment that boosts PD-1⁺ lymphocytes, exacerbate autoimmune disorders (Salama, A. D., et al., J Exp Med, 2003. 198(1); p. 71-8; Godwin, J. L., et al., J Immunother Cancer, 2017. 5: p. 40; Ansari, M. J., et al., J Exp Med, 2003. 198(1); p. 63-9; and Hughes, J., et al., Diabetes Care, 2015. 38(4); p, e55-7). Based on these observations, PD-1⁺ cells may be largely viewed as pathogenic cells of autoimmune disease. Consequently, the suppression and depletion of PD-1⁺ cells may be an effective strategy to ameliorate autoimmune disease. Recent research has proven therapeutic effects of the depletion of PD-1⁺ cells in mouse models of experimental autoimmune encephalomyelitis (EAE) and Type-1 diabetes (Zhao, P., et al., Nat Biomed Eng, 2019. 3(4); p. 292-305).

PD-1⁺ cells are also highlighted in cancer immunotherapy. PD-1⁺ T cells are believed to be major effector cells in the success of PD-1 immune checkpoint therapy (Ivashko, I. N. and J. M. Kolesar, Am J Health Syst Pharm, 2016. 73(4); p. 193-201; and Derosiers, N., et al., J Immunol, 2022. 208(2); p. 278-28). However, PD-1⁺ Tregs and PD-1⁺ cancer cells may also be involved in resistance to PD-1 immune checkpoint therapy: the resistance is indeed common among patients who receive the therapy (Borcherding, N., et al., J Mol Biol, 2018. 430(14); p. 2014-2029; Xu-Monette, et al., Blood, 2018. 131(1); p. 68-83; Lesokhin, A. M., et al., J Clin Oncol, 2016. 34(23); p. 2698-704; Ratner, L., et al., N Engl J Med, 2018. 378(20): p. 1947-1948; Robert, C., et al., J Clin Oncol, 2020. 38(33); p. 3937-3946; and Hamid, O., et al., Ann Oncol, 2019. 30(4); p. 582-588). Tumor cells that intrinsically express PD-1 were recently discovered in many types of tumors (Xu-Monette, Z. Y., et al., Blood, 2018. 131(1): p. 68-83; Wang, X., et al., Proc Natl Acad Sci USA, 2020. 117(12); p. 6640-6650; Kleffel, S., et al., Cell, 2015. 162(6); p. 1242-56; Yao, H., et al., Front Immunol, 2018. 9: p. 1774; Du. S., et al., Oncoimmunology, 2018. 7(4); p, e1408747; and Schatton, T., et al., Cancer Res, 2010. 70(2); p. 697-708). These cancer cells were proposed to play roles in tumorigenesis and the resistance to the PD-1 immune checkpoint therapy (Wang, X., et al., Proc Natl Acad Sci USA, 2020. 117(12); p. 6640-6650; Kleffel, S., et al., Cell, 2015. 162(6); p. 1242-56; Yao, H., et al., Front Immunol, 2018. 9: p. 1774; Du, S., et al., Oncoimmunology, 2018. 7(4); p, e1408747; Li, H., et al., Hepatology, 2017. 66(6); p. 1920-1933; Wartewig, T., et al., Nature, 2017. 552(7683); p. 121-125). However, apparently competing views exist regarding the relationship between these cells and the immune checkpoint blockade. According to one side of the view, PD-1 on tumor cell functions as a tumor suppressor, and theoretically, a blockade of the PD-1 results in a promotion of tumor growth (Wang, X., et al., Proc Natl Acad Sci USA. 2020. 117(12); p. 6640-6650). The other side of the view, however, believes that tumor cell-intrinsic PD-1 acts to facilitate tumor growth (Kleffel, S., et al., Cell, 2015. 162(6); p. 1242-56) and that PD-1⁺ tumor cells are tumor initiating cells (Schatton, T., et al., Cancer Res, 2010. 70(2); p. 697-708). Such contradictory views may be partially attributed to the research approaches through which these conclusions were reached: the PD-1 blockade, PD-1 over-expression, and PD-1 knockout (Wang, X., et al., Proc Natl Acad Sci USA, 2020. 117(12); p. 6640-6650; Kleffel, S., et al., Cell, 2015. 162(6); p. 1242-56; Du, S., et al., Oncoimmunology, 2018. 7(4); p, e1408747; and Li. H., et al., Hepatology, 2017. 66(6); p. 1920-1933). These approaches would not be able to reveal the impacts of PD-1⁺ tumor cells as a whole, specifically regarding the tumorigenesis and resistance to the PD-1 immune checkpoint therapy. As such, the depletion of PD-1⁺ tumor cells may have an impact of the PD-1 immune checkpoint therapy.

Previously, an immunotoxin that specifically targeted PD-1⁺ cells was generated (Zhao, P., et al., Nat Biomed Eng, 2019. 3(4); p. 292-305). For mice with EAE and type-1 diabetes, the treatment of the PD-1 immunotoxin reduced PD-1⁺ lymphocytes in these mice and ameliorated their autoimmune diseases. On the other hand, the administration of the PD-1 immunotoxin did not compromise healthy adaptive immunity, evidenced by full-strength immune responses to vaccinations in treated mice to vaccinations. Depleting antibodies have been used clinically for a wide range of diseases with proven safety, scalability, and industrial feasibility. Another advantage of antibodies is their long plasma half-lives (10-21 days in human) due to their high affinity binding with FcRn (neonatal Fc receptor) and subsequent escape of endosomal degradation (Booth, B. J., et al., MAbs, 2018. 10(7); p. 1098-1110). Depleting antibodies may cause the elimination of the cells that express corresponding antigens by Fc-mediated effector mechanisms including antibody-dependent cell-mediated cytotoxicity (ADCC), complement dependent cytotoxicity (CDC) and antibody dependent cell-mediated phagocytosis (ADCP). CDC and ADCP relies on the binding between depleting antibodies and FcγR on effector cells such as macrophages (Stewart, R., et al., Journal for Immuno Therapy of Cancer, 2014. 2(1); p. 29). Mouse FcγRIV is one type of activation FcR expressed on macrophages, monocytes, and neutrophils (Bruhns, P., Blood, 2012. 119(24); p. 5640-5649) and has been chosen as a target during the development of mouse depleting antibodies (Simpson, T. R., et al., J Exp Med, 2013. 210(9); p. 1695-710). Among the mouse IgG subtypes. IgG2a and IgG2b antibodies show the highest binding affinity with the FcγRIV (Bruhns, P., Blood, 2012. 119(24); p. 5640-5649). Different from IgG2b ones, mouse IgG2a antibodies also has a high binding affinity with mouse FcγRI, which expresses on monocytic dendritic cells and contributes to depleting effect of the antibodies (León, B., et al, Semin Immunol, 2005. 17(4); p. 313-8). Additionally, mouse IgG2a antibodies have long plasma half-lives as of 5-8 days (Brunn, N. D., et al., J Pharmacol Exp Ther, 2016. 356(3); p. 574-86; Deng, R., et al., MAbs, 2012. 4(1); p. 101-9; Ghetie, V., et al., Pharmacokinetics of Antibodies and Immunotoxins in Mice and Humans, in Handbook of Anticancer Pharmacokinetics and Pharmacodynamics, W. D. Figg and H. L. McLeod, Editors. 2004. Humana Press: Totowa, NJ. p. 475-498; and Vieira. P. and K. Rajewsky. Eur J Immunol, 1988. 18(2); p. 313-6). Thus, it is desirable to engineer mouse depleting antibodies of the IgG2a subtype.

Disclosed herein are antibodies that were engineered and generated to deplete PD-1⁺ cells (referred to herein as D-αPD-1 antibodies). D-αPD-1 antibodies consist of variable domains of a known anti-mouse PD-1 antibody (clone RMP1-14, termed B-αPD-1 hereafter) and constant domains from mouse IgG2a heavy chain and K-light chain. D-αPD-1 antibodies bind specifically to PD-1⁺ cells. Importantly, D-αPD-1 antibodies are able to eliminate PD-1⁺ cells in vivo. Lastly, D-αPD-1 antibodies were found to utilize CDC and ADCP mechanisms to abolish PD-1⁺ cells, to the D-αPD-1 antibodies described herein can deplete PD-1⁺ cells and be useful in the treatment and management of cancer and autoimmune diseases.

Although the foregoing disclosure has been described in some detail by way of illustration and example for purposes of clarity of understanding, certain changes and modifications may be practiced within the scope of the appended claims.

### Compositions

Anti-PD-1 antibodies. Disclosed herein are isolated antibodies, including, but not limited to, anti-PD-1 antibodies or binding fragments thereof. Disclosed herein are anti-PD-1 antibodies or binding fragments thereof that bind to PD-1 on T cells. Disclosed herein are anti-PD-1 antibodies or binding fragments thereof that bind to PD-1 and block or inhibit the immune suppressive function of the PD-1/PD-L1 interaction (e.g., block or inhibit the binding of PD-1 to PD-L1). Disclosed herein are anti-PD-1 antibodies or binding fragments thereof useful in treating autoimmune disorders. Also disclosed herein are anti-PD-1 antibodies or binding fragments thereof useful in the treating cancer and inhibiting or preventing tumor or cancer metastases.

The anti-PD-1 antibodies disclosed herein can be of the IgG, IgM, IgA, IgD, and IgE Ig classes, as well as polypeptides comprising one or more antibody CDR domains that retain antigen binding activity. Illustratively, the anti-PD-1 antibodies may be chimeric, affinity matured, humanized, or human antibodies. In some aspects, the anti-PD-1 antibodies can be monoclonal antibodies. In some aspects, the monoclonal anti-PD-1 antibody can be a humanized antibody. By known means and as described herein, polyclonal or monoclonal antibodies, antibody fragments, binding domains and CDRs (including engineered forms of any of the foregoing) may be created that are specific for PD-1 antigen, one or more of its respective epitopes, or conjugates of any of the foregoing, whether such antigens or epitopes are isolated from natural sources or are synthetic derivatives or variants of the natural protein.

Also disclosed herein are compositions comprising the disclosed isolated antibodies, including, but not limited to anti-PD-1 antibodies. In some aspects, the antibodies disclosed herein can be isolated antibodies. Examples of the CDR sequences and heavy or light chain variable region sequences of anti-PD-1 antibodies are shown in Table 3.

In some aspects, complementarity determining regions (CDRs) of the heavy chain or light chain of an anti-PD-1 antibody can be used to prepare an anti-PD-1 antibody or a fragment thereof (e.g. scFv). For example, disclosed herein are anti-PD-1 antibodies comprising one or more of CDRs including CDRs of the heavy chain: SSYRWN (SEQ ID NO: 1), YINSAGISNYNPSLKR (SEQ ID NO: 2), and SDNMGTTPFTY (SEQ ID NO: 3); or CDRs of the light chain: RSSKSLLYSDGKTYLN (SEQ ID NO: 4), WMSTRAS (SEQ ID NO: 5), and QQGLEFPT (SEQ ID NO: 6).

Disclosed herein are anti-PD-1 antibodies comprising mutations in the V_{H} and V_{L}, respectively, of an αPD-1 antibody. For example, Table 3 provides an example of two mutations (underlined) wherein the mutations are V_{H}: R45C; V_{L}: G104C. In some aspects, disclosed herein are antibodies or antigen-binding portions thereof, comprising: a heavy chain sequence and a light chain sequence, wherein the heavy chain sequence comprises SEQ ID NOs: 7. 9, 11 or 12 and wherein the light chain sequence comprises SEQ ID NOs: 8, 10, 13, 14 or 15. In some aspects, the heavy and light chain sequences can exhibit a certain degree of identity or homology to the SEQ ID NOS: 7, 8, 9, 10, 11, 12, 13, 14, or 15. The degree of identity can vary and be determined by methods known to one of ordinary skill in the art.

The terms “homology” and “identity” each refer to sequence similarity between two polypeptide sequences. The heavy and light chain sequences of an anti-PD-1 antibody comprising one or more mutations V_{H} and V_{L}, respectively of an αPD-1 as described herein can have at least or about 25%, 50%, 65%, 75%, 80%, 85%, 90%, 95%, 96%, 97%, 98%, or 99% identity or homology to SEQ ID NOs: 7, 8, 9, 10, 11, 12, 13. 14, and/or 15.

In some aspects, one or more of the heavy or light chain CDR sequences can comprise at least one substitution or at least one amino acid substitution compared to the parent heavy or light chain sequence (e.g., SEQ ID Nos: 7, 9, 11, or 12; or 8, 10, 13, 14, or 15, respectively). In some aspects, one or more of the heavy or light chain CDR sequences can comprise at least one substitution or at least one amino acid substitution compared to the parent CDR (e.g., SEQ ID Nos: 1, 2, 3, 4, 5 or 6).

Disclosed herein are anti-PD-1 antibodies comprising mutations in the V_{H} and V_{L}, respectively of an αPD-1. For example, two mutations can be V_{H}: R45C; V_{L}: G104C. In some aspects, disclosed herein are antibodies or antigen-binding portions thereof, comprising: a heavy chain sequence and a light chain sequence, wherein the heavy chain sequence comprises SEQ ID NOs: 7. 9, 11, or 12 and wherein the light chain sequence comprises SEQ ID NOs: 8, 10, 13, 14 or 15, and wherein the antibody comprises one or more of CDRs selected from the group of SSYRWN (SEQ ID NO: 2), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

Disclosed herein are antibodies or antigen-binding portion thereof, comprising: a heavy chain sequence and a light chain sequence, wherein the heavy chain sequence comprises SEQ ID NOs: 7. 9, 11, or 12 and wherein the light chain sequence comprises SEQ ID NOs: 8, 10, 13, 14 or 15, and wherein the antibody comprises one or more of CDRs selected from the group of SSYRWN (SEQ ID NO: 1), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

Disclosed herein are antibodies or antigen-binding portion thereof, comprising: a heavy chain sequence and a light chain sequence, wherein the heavy chain sequence consists of SEQ ID NOs: 7, 9, 11, or 12 and wherein the light chain sequence consists of SEQ ID NOs: 8. 10. 13, 14 or 15.

Disclosed herein are antibodies or antigen-binding portion thereof, comprising: a heavy chain sequence and a light chain sequence, wherein the heavy chain sequence consists of SEQ ID NOs: 7, 9, 11, or 12 and wherein the light chain sequence consists of SEQ ID NOs: 8. 10. 13, 14 or 15, and wherein the antibody comprises one or more of CDRs selected from the group of SSYRWN (SEQ ID NO: 1). YINSAGISNYNPSLKR (SEQ ID NO: 2). SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

Disclosed herein are antibodies or antigen-binding portion thereof, comprising: a heavy chain sequence and a light chain sequence, wherein the heavy chain sequence consists of SEQ ID NOs: 7, 9, 11, or 12 and wherein the light chain sequence consists of SEQ ID NOs: 8. 10. 13, 14 or 15, and wherein the antibody comprises one or more of CDRs selected from the group of SSYRWN (SEQ ID NO: 1). YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

As described herein, SEQ ID NOs: 7. 9, 11, and 12 are examples of heavy chain sequences and SEQ ID NOs: 8, 10, 13, 14 or 15 are examples of light chain sequences.

In some aspects, the scFv can be from an anti-PD-1 antibody comprising mutations in the V_{H} and V_{L}, respectively of an αPD-1. An example of two are V_{H}: R45C; V_{L}: G104C. In some aspects, disclosed herein is an antibody or antigen-binding portion thereof, comprising: a heavy chain sequence and a light chain sequence, wherein the heavy chain sequence comprises SEQ ID NOs: 7, 9, 11, or 12 and wherein the light chain sequence comprises SEQ ID NOs: 8, 10, 13, 14 or 15, and wherein the antibody comprises one or more of CDRs selected from the group of SSYRWN (SEQ ID NO: 1), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

In some aspects, the scFv can be from an antibody or antigen-binding portion thereof, comprising: a heavy chain sequence and a light chain sequence, wherein the heavy chain sequence comprises SEQ ID NOs: 7, 9, 11, or 12 and wherein the light chain sequence comprises SEQ ID NOs: 8, 10, 13, 14 or 15, and wherein the antibody comprises one or more of CDRs selected from the group of SSYRWN (SEQ ID NO: 1), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

In some aspects, the scFv can be from antibody or antigen-binding portion thereof, comprising: a heavy chain sequence and a light chain sequence, wherein the heavy chain sequence consists of SEQ ID NOs: 7, 9, 11, or 12 and wherein the light chain sequence consists of SEQ ID NOs: 8, 10, 13, 14 or 15.

In some aspects, the scFv can be from antibody or antigen-binding portion thereof, comprising: a heavy chain sequence and a light chain sequence, wherein the heavy chain sequence consists of SEQ ID NOs: 7, 9, 11 or 12 and wherein the light chain sequence consists of SEQ ID NOs: 8, 10, 13, 14 or 15, and wherein the antibody comprises one or more of CDRs selected from the group of SSYRWN (SEQ ID NO: 1), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

In some aspects, the scFv can be from antibody or antigen-binding portion thereof, comprising: a heavy chain sequence and a light chain sequence, wherein the heavy chain sequence consists of SEQ ID NOs: 7. 9. 11 or 12 and wherein the light chain sequence consists of SEQ ID NOs: 8, 10, 13, 14 or 15, and wherein the antibody comprises one or more of CDRs selected from the group of SSYRWN (SEQ ID NO: 1), YINSAGISNYNPSLKR (SEQ ID NO: 2), SDNMGTTPFTY (SEQ ID NO: 3), RSSKSLLYSDGKTYLN (SEQ ID NO: 4), WMSTRAS (SEQ ID NO: 5), and QQGLEFPT (SEQ ID NO: 6).

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 4; a complementarity determining region light chain 2 (CDRL2) amino acid sequence of SEQ ID NO: 5; and a complementarity determining region light chain 3 (CDRL3) amino acid sequence of SEQ ID NO: 6. In some aspects, the heavy chain variable region can comprise a complementarity determining region heavy chain 1 (CDRH1) amino acid sequence of SEQ ID NO: 1; a complementarity determining region heavy chain 2 (CDRH2) amino acid sequence of SEQ ID NO: 7, 9, 11, or 12; and a complementarity determining region heavy chain 3 (CDRH3) amino acid sequence of SEQ ID NO: 8, 10, 13, 14 or 15.

Also disclosed herein, are isolated antibodies comprising a light chain variable region amino acid sequence of SEQ ID NO: 7, 9, 11, or 12 and a heavy chain variable region amino acid sequence of SEQ ID NO: 8, 10, 13, 14 or 15.

In some aspects, any of the antibodies disclosed herein can comprise a light chain variable region amino acid sequence comprising SEQ ID NO: 7, 9, 11, or 12. In some aspects, any of the antibodies disclosed herein can comprise a heavy chain variable region amino acid sequence comprising SEQ ID NO: 8, 10, 13. 14 or 15. In some aspects, a light chain variable region has an amino acid sequence that is at least 90% identical to amino acid sequence SEQ ID NO: 7. 9, 11, or 12. In some aspects, a heavy chain variable region has an amino acid sequence that is at least 90% identical to amino acid sequence SEQ ID NO: 8, 10, 13, 14 or 15.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 4; a determining region light chain 2 (CDRL2) amino acid sequence of SEQ ID NO: 5; and a determining region light chain 3 (CDRL3) amino acid sequence of SEQ ID NO: 6; and wherein the heavy chain variable region comprises a complementarity determining region heavy chain 1 (CDRH1) amino acid sequence of SEQ ID NO: 1; a complementarity determining region heavy chain 2 (CDRH2) amino acid sequence of SEQ ID NO: 2; and a complementarity determining region heavy chain 3 (CDRH3) amino acid sequence of SEQ ID NO: 3, wherein one or more of the CDRL1, CDRL2. CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 8, 10, 13, 14 or 15 and a heavy chain variable region amino acid sequence of SEQ ID NO: 7, 9, 11, or 12, wherein the isolated antibody comprises 1, 2, 3, 4, or 5 conservative amino acid substitutions in the light or heavy chain variable region amino acid sequences.

TABLE 3
Exemplary Amino Acid Sequences of anti-PD1 antibody

| SEQ ID | | |
|---|---|---|
| NO: | Sequence | Name |
| 1 | SSYRWN | complementarity |
| | | determining region |
| | | heavy chain 1 |
| 2 | YINSAGISNYNPSLKR | complementarity |
| | | determining region |
| | | heavy chain 2 |
| 3 | SDNMGTTPFTY | complementarity |
| | | determining region |
| | | heavy chain 3 |
| 4 | RSSKSLLYSDGKTYLN | complementarity |
| | | determining region |
| | | light chain 1 |
| 5 | WMSTRAS | a complementarity |
| | | determining region |
| | | light chain 2 |
| 6 | QQGLEFPT | a complementarity |
| | | determining region |
| | | light chain 3 |
| 7 | EVQLQESGPGLVKPSQSLSLTCSVTGYSI | anti-PD-1 heavy |
| | TSSYRWNWIRKFPGNCLEWMGYINSAG | chain variable |
| | ISNYNPSLKRRISITRDTSKNQFFLQVNSV | domain |
| | TTEDAATYYCARSDNMGTTPFTYWGQG | |
| | TLVTVSS | |
| 8 | DIVMTQGTLPNPVPSGESVSITCRSSKSL | anti-PD-1 light chain |
| | LYSDGKTYLNWYLQRPGQSPQLLIYW | variable domain |
| | MSTRASGVSDRFSGSGSGTDFTLKISG | |
| | VEAEDVGIYYCQQGLEFPTFGCGTKLELK | |
| 9 | EVQLQESGPGLVKPSQSLSLTCSVTGYSI | anti-PD-1 heavy |
| | TSSYRWNWIRKFPGNRLEWMGYINSAG | chain variable |
| | ISNYNPSLKRRISITRDTSKNQFFLQVNSV | domain |
| | TTEDAATYYCARSDNMGTTPFTYWGQG | |
| | TLVTVSS | |
| 10 | DIVMTQGTLPNPVPSGESVSITCRSSKSL | anti-PD-1 light chain |
| | LYSDGKTYLNWYLQRPGQSPQLLIYW | variable domain |
| | MSTRASGVSDRFSGSGSGTDFTLKISG | |
| | VEAEDVGIYYCQQGLEFPTFGGGTKLELK | |
| 11 | MRMLVLLYLLTALPGILSEVQLQESGPGL | anti-PD-1 heavy |
| | VKPSQSLSLTCSVTGYSITSSYRWNWIRK | chain amino acid |
| | FPGNRLEWMGYINSAGISNYNPSLKRRIS | sequence; anti-PD-1 |
| | ITRDTSKNQFFLQVNSVTTEDAATYYCA | antibody (aPD-1, |
| | RSDNMGTTPFTYWGQGTLVTVSSAKTT | mouse IgG2a) bolded |
| | APSVYPLAPVCGDTTGSSVTLGCLVKGY | sequence includes |
| | FPEPVTLTWNSGSLSSGVHTFPAVLQSDL | signal sequence |
| | YTLSSSVTVTSSTWPSQSITCNVAHPASS | |
| | TKVDKKIEPRGPTIKPCPPCKCPAPNLLG | |
| | GPSVFIFPPKIKDVLMISLSPIVTCVVVDV | |
| | SEDDPDVQISWFVNNVEVHTAQTQTHRE | |
| | DYNSTLRVVSALPIQHQDWMSGKEFKCK | |
| | VNNKDLPAPIERTISKPKGSVRAPQVYVL | |
| | PPPEEEMTKKQVTLTCMVTDFMPEDIYV | |
| | EWTNNGKTELNYKNTEPVLDSDGSYFM | |
| | YSKLRVEKKNWVERNSYSCSVVHEGLH | |
| | NHHTTKSFSRTPGK | |
| 12 | EVQLQESGPGLVKPSQSLSLTCSVTGYSI | anti-PD-1 heavy |
| | TSSYRWNWIRKFPGNRLEWMGYINSAG | chain amino acid |
| | ISNYNPSLKRRISITRDTSKNQFFLQVNSV | sequence; anti-PD-1 |
| | TTEDAATYYCARSDNMGTTPFTYWGQG | antibody (aPD-1, |
| | TLVTVSSAKTTAPSVYPLAPVCGDTTGSS | mouse IgG2a) |
| | VTLGCLVKGYFPEPVTLTWNSGSLSSGV | without the signal |
| | HTFPAVLQSDLYTLSSSVTVTSSTWPSQS | sequence |
| | ITCNVAHPASSTKVDKKIEPRGPTIKPCPP | |
| | CKCPAPNLLGGPSVFIFPPKIKDVLMISLS | |
| | PIVTCVVVDVSEDDPDVQISWFVNNVEV | |
| | HTAQTQTHREDYNSTLRVVSALPIQHQD | |
| | WMSGKEFKCKVNNKDLPAPIERTISKPK | |
| | GSVRAPQVYVLPPPEEEMTKKQVTLTCM | |
| | VTDFMPEDIYVEWTNNGKTELNYKNTEP | |
| | VLDSDGSYFMYSKLRVEKKNWVERNSY | |
| | SCSVVHEGLHNHHTTKSFSRTPGK | |
| 13 | MRCSLQFLGLLVLWIPGLNGDIVMTQGTLP | anti-PD-1 kappa light |
| | NPVPSGESVSITCRSSKSLLYSDGKTYLNWY | chain amino acid |
| | LQRPGQSPQLLIYWMSTRASGVSDRFSGSG | sequence with signal |
| | SGTDFTLKISGVEAEDVGIYYCQQGLEFPTF | sequence (bold) |
| | GGGTKLELKRADAAPTVSIFPPSSEQLTSGG | |
| | ASVVCFLNNFYPKDINVKWKIDGSERQNGV | |
| | LNSWTDQDSKDSTYSMSSTLTLTKDEYERH | |
| | NSYTCEATHKTSTSPIVKSFNRNEC | |
| 14 | GDIVMTQGTLPNPVPSGESVSITCRSSK | anti-PD-1 kappa light |
| | SLLYSDGKTYLNWYLQRPGQSPQLLIYW | chain amino acid |
| | MSTRASGVSDRFSGSGSGTDFTLKISGVE | sequence; without the |
| | AEDVGIYYCQQGLEFPTFGGGTKLELKR | signal sequence |
| | ADAAPTVSIFPPSSEQLTSGGASVVCFLN | |
| | NFYPKDINVKWKIDGSERQNGVLNSWTD | |
| | QDSKDSTYSMSSTLTLTKDEYERHNSYT | |
| | CEATHKTSTSPIVKSFNRNEC | |
| 15 | DIVMTQGTLPNPVPSGESVSITCRSSK | anti-PD-1 kappa light |
| | SLLYSDGKTYLNWYLQRPGQSPQLLIYW | chain amino acid |
| | MSTRASGVSDRFSGSGSGTDFTLKISGVE | sequence; without the |
| | AEDVGIYYCQQGLEFPTFGGGTKLELKR | signal sequence |
| | ADAAPTVSIFPPSSEQLTSGGASVVCFLN | |
| | NFYPKDINVKWKIDGSERQNGVLNSWTD | |
| | QDSKDSTYSMSSTLTLTKDEYERHNSYT | |
| | CEATHKTSTSPIVKSFNRNEC | |

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3, 4 or 5 amino acid changes as compared to the parent or reference CDR of the invention, and shares or improves biological function, specificity and/or activity of the parent CDR.

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar-alanine, valine, leucine, isoleucine, proline, phenylalanine, methionine, tryptophan; and (4) uncharged polar glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, an isolated replacement of a leucine with an isoleucine or valine, an aspartate with a glutamate, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are as described in Table 1, supra. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

In some aspects, the CDRs can be defined according to the Kabut definition. In some aspects, the CDRs can be defined according to the IMGT definition.

In some aspects, the antibodies disclosed herein can be recombinantly engineered, chimerized, or humanized. In some aspects, the antibodies disclosed herein can be affinity matured or human antibodies. In some aspects, the antibodies disclosed herein can be a Fab, an Fab′, an F(ab)2, a Fv, a scFv, a diabody or fragments thereof. In some aspects, the antibody can be a monoclonal antibody. In some aspects, the monoclonal antibodies can be humanized or chimeric forms thereof. In some aspects, the monoclonal antibody can be a humanized antibody. By known means and as described herein, polyclonal or monoclonal antibodies, antibody fragments, binding domains and CDRs (including engineered forms of any of the foregoing) may be created that are specific for PD-1 antigen, one or more of its respective epitopes, or conjugates of any of the foregoing, whether such antigens or epitopes are isolated from natural sources or are synthetic derivatives or variants of the natural protein.

A monoclonal antibody is a single, clonal species of antibody wherein every antibody molecule recognizes the same epitope because all antibody producing cells are derived from a single, antibody-producing B-lymphocyte (or other clonal cell, such as a cell that recombinantly expresses the antibody molecule). The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions. Hybridoma technology as used in monoclonal antibody production involves the fusion of a single, antibody-producing B lymphocyte isolated from a mouse previously immunized with a CCL21 protein or peptide with an immortalized cell, e.g., a mouse cell line. This technology provides a method to propagate a single antibody-producing cell for an indefinite number of generations, such that unlimited quantities of structurally identical antibodies having the same antigen or epitope specificity, i.e., monoclonal antibodies, may be produced.

Methods have been developed to replace light and heavy chain constant domains of the monoclonal antibody with analogous domains of human origin, leaving the variable regions of the foreign antibody intact. Alternatively, “fully human” monoclonal antibodies are produced in mice or rats that are transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDRs are derived from non-human (e.g., mouse, rat, chicken, llama, etc.) monoclonal antibodies, and the framework regions are derived from human antibody amino acid sequences. The replacement of amino acid sequences in the antibody that are characteristic of rodents with amino acid sequences found in the corresponding positions of human antibodies reduces the likelihood of adverse immune reaction to foreign protein during therapeutic use in humans. A hybridoma or other cell producing an antibody may also be subject to genetic mutation or other changes, which may or may not alter the binding specificity of antibodies produced by the hybridoma.

Engineered antibodies may be created using monoclonal and other antibodies and recombinant DNA technology to produce other antibodies or chimeric molecules that retain the antigen or epitope binding specificity of the original antibody. i.e., the molecule has a specific binding domain. Such techniques may involve introducing DNA encoding the immunoglobulin variable region or the CDRs of an antibody into the genetic material for the framework regions, constant regions, or constant regions plus framework regions, of a different antibody. See, for instance, U.S. Pat. Nos. 5,091,513 and 6,881,557, which are incorporated herein by reference.

By known means as described herein, polyclonal or monoclonal antibodies, antibody fragments having binding activity, binding domains and CDRs (including engineered forms of any of the foregoing), may be created that specifically bind to PD-1 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. In some aspects, the antibodies can be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in Marks et al., 1992, Bio/Technol., 10:779-783; Stemmer, 1994, Nature, 370:389-391; Gram et al., 1992, Proc. Natl. Acad. Sci. USA, 89:3576-3580; Barbas et al., 1994, Proc. Natl. Acad. Sci. USA, 91:3809-3813; and Schier et al., 1996, Gene, 169(2); 147-155.

Methods for producing polyclonal antibodies in various animal species, as well as for producing monoclonal antibodies of various types, including humanized, chimeric, and fully human, are well known in the art and are highly reproducible. For example, the following U.S. patents provide descriptions of such methods and are herein incorporated by reference: U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; 7,407,659; and 8,178,098.

In some aspects, the antibody can be a single chain antibody. In some aspects, the antibody can be linked to a detectable label. In some aspects, antibody can be a monovalent or a bivalent antibody.

In some aspects, the antibodies disclosed herein can be an IgG, an IgM, an IgA, an IgD, or an IgE antibody or antigen binding fragment thereof. In some aspects, the antibodies can be of the IgG, IgM, IgA, IgD, and IgE Ig classes or a genetically modified IgG class antibody, as well as polypeptides comprising one or more antibody CDR regions that retain antigen binding activity. In some aspects, the antibody can be an IgG class of antibody. In some aspects, the IgG class antibody can be an IgG1, IgG2, IgG3, or IgG4 class antibody.

In some aspects, the antibody can be a bispecific antibody. Unifying two antigen binding sites of different specificity into a single construct, bispecific antibodies have the ability to bring together two discreet antigens with exquisite specificity and therefore have great potential as therapeutic agents. Bispecific antibodies were originally made by fusing two hybridomas, each capable of producing a different immunoglobulin. Bispecific antibodies can also be produced by joining two scFv antibody fragments while omitting the Fc portion present in full immunoglobulins. Each scFv unit in such constructs can contain one variable domain from each of the heavy (V_{H}) and light (V_{L}) antibody chains, joined with one another via a synthetic polypeptide linker, the latter often being genetically engineered so as to be minimally immunogenic while remaining maximally resistant to proteolysis. Respective scFv units may be joined by a number of known techniques, including incorporation of a short (usually less than 10 amino acids) polypeptide spacer bridging the two scFv units, thereby creating a bispecific single chain antibody. The resulting bispecific single chain antibody is therefore a species containing two V_{H}/V_{L} pairs of different specificity on a single polypeptide chain, in which the V_{H} and V_{L} domains in a respective scFv unit are separated by a polypeptide linker long enough to allow intramolecular association between these two domains, such that the so-formed scFv units are contiguously tethered to one another through a polypeptide spacer kept short enough to prevent unwanted association between, for example, the V_{H} domain of one scFv unit and the V_{L} of the other scFv unit.

Examples of antibody fragments suitable for use include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), in which a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent, or multispecific fragments constructed by gene fusion (U.S. Patent Appln. Pub. No. 20050214860). Fv, scFv, or diabody molecules may be stabilized by the incorporation of disulfide bridges linking the V_{H} and V_{L} domains. Minibodies comprising a scFv joined to a C_{H3} domain (Hu et al., 1996, Cancer Res., 56:3055-3061) may also be useful. In addition, antibody-like binding peptidomimetics are also contemplated. “Antibody like binding peptidomimetics” (ABiPs), which are peptides that act as pared-down antibodies and have certain advantages of longer serum half-life as well as less cumbersome synthesis methods, have been reported by Liu et al., 2003, Cell Mol. Biol., 49:209-216.

Animals may be inoculated with an antigen, such as a PD-1 polypeptide or peptide to generate an immune response and produce antibodies specific for the PD-1 polypeptide. Frequently, an antigen is bound or conjugated to another molecule to enhance the immune response. As used herein, a conjugate can be any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The antibodies described herein directed to PD-1 will have the ability to neutralize, block, inhibit, or counteract the effects of PD-1 binding to PD-L1 regardless of the animal species, monoclonal cell line or other source of the antibody. Certain animal species may be less preferable for generating therapeutic antibodies because they may be more likely to cause an immune or allergic response due to activation of the complement system through the “Fc” portion of the antibody. However, whole antibodies may be enzymatically digested into the “Fc” (complement binding) fragment, and into peptide fragments having the binding domains or CDRs. Removal of the Fc portion reduces the likelihood that this antibody fragment will elicit an undesirable immunological response and, thus, antibodies without an Fc portion may be preferential for prophylactic or therapeutic treatments. As described above, antibodies may also be constructed so as to be chimeric, humanized, or partially or fully human, so as to reduce or eliminate potential adverse immunological effects resulting from administering to an animal an antibody that has been produced in, or has amino acid sequences from, another species.

In some aspects, the antibody binds to PD-1 with an equilibrium dissociation constant (K_{D}) of less than or equal to 50 nM (the smaller the K_{D} value, the stronger the binding affinity). In some aspects, the antibody selectively binds to PD-1 and inhibits binding of PD-1 to PD-L1. In some aspects, the antibody selectively binds to human PD-1 and inhibits binding of human PD-1 to human PD-L1. In some aspects, the anti-PD-1 antibody selectively binds to human PD-1 positive cells and the antibody bound to the human PD-1 positive cells can be depleted via antibody-dependent cellular phagocytosis and complement dependent cytotoxicity.

The term “specifically binds” (or “immunospecifically binds”) is not intended to indicate that an antibody binds exclusively to its intended target. Rather, an antibody “specifically binds” if its affinity for its intended target is about, for example, 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10-¹⁰M⁻¹ to about 10¹¹ M⁻¹. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the antibodies described herein comprise a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 1, SEQ ID NO: 2, and SEQ ID NO: 3, respectively; and/or a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 4, SEQ ID NO: 5, and SEQ ID NO: 6, respectively.

In some aspects, the antibodies disclosed herein can specifically bind to an epitope of PD-1. In some aspects, the antibodies disclosed herein can specifically bind to an area of PD-1 wherein the intrinsic ligands of PD-1, PD-L1 and/or PD-L2 bind.

In some aspects, the antibodies disclosed herein can prevent, inhibit or block PD-1 binding to PD-L1. In some aspects, the antibodies disclosed herein can inhibit the binding of human PD-1 to human PD-L1. In some aspects, the antibodies disclosed herein bind to cells expressing PD-1. In some aspects, the antibodies disclosed herein can, for example, bind to tumor cells that express PD-1 on their surface. The association of the antibodies disclosed herein leads to immune effector responses that then lead to destruction of the tumor cells that express PD-1 and are bound to the disclosed antibodies, thereby overcoming or avoiding tumor's resistance to cancer immunocheckpoint therapy. For autoimmune disease, the antibodies disclosed herein can, for example, bind to pathogenic PD-1 expressing immune cells and lead to destrcution of these immune cells. The destruction of these pathogenic immune cells provides a curative benefit to autoimmune disease patients.

Antibody proteins may be recombinant, or synthesized in vitro. It is contemplated that in anti-PD-1 antibody-containing compositions as described herein can comprise between about 0.001 mg and about 10 mg of total antibody polypeptide per ml. Thus, the concentration of antibody protein in a composition can be about, at least about or at most about or equal to 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, at most about, or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42. 43, 44, 45, 46, 47, 48, 49, 50, 51, 52, 53, 54, 55, 56, 57, 58, 59, 60, 61, 62, 63, 64, 65, 66, 67. 68, 69, 70, 71, 72, 73, 74, 75, 76, 77, 78, 79, 80, 81, 82, 83, 84, 85, 86, 87, 88, 89, 90, 91, 92. 93, 94, 95, 96, 97, 98, 99, or 100% may be an antibody that binds PD-1.

Disclosed herein are compositions comprising any of the antibodies or isolated antibodies described herein. In some aspects, the compositions can further comprise at least one pharmaceutically acceptable carrier or diluent.

In some aspects, the compositions described herein can comprise a detectable label or reporter. An antibody or an immunological portion of an antibody that retains binding activity, can be chemically conjugated to, or recombinantly expressed as, a fusion protein with other proteins. For the purposes as described herein, all such fused proteins are included in the definition of antibodies or an immunological portion of an antibody. In some aspects, antibodies and antibody-like molecules generated against PD-1 or polypeptides that are linked to at least one agent to form an antibody conjugate or payload are encompassed. In order to increase the efficacy of antibody molecules as diagnostic or therapeutic agents, it is conventional to link or covalently bind or complex at least one desired molecule or moiety to the antibody. Such a linked molecule or moiety may be, but is not limited to, at least one effector, detectable label or reporter molecule. Effector molecules comprise molecules having a desired activity, e.g., cytotoxic activity. Non-limiting examples of effector molecules that may be attached to antibodies include toxins, therapeutic enzymes, antibiotics, radio-labeled nucleotides and the like. By contrast, a reporter molecule or detectable label is defined as any moiety that may be detected using an assay. Non-limiting examples of reporter molecules and detectable labels that can be conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin, and the like. Several methods are known in the art for attaching or conjugating an antibody to a conjugate molecule or moiety. Some attachment methods involve the use of a metal chelate complex, employing by way of nonlimiting example, an organic chelating agent such a diethylenetriaminepentaacetic acid anhydride (DTPA); ethylenetriaminetetraacetic acid; N-chloro-p-toluenesulfonamide; and/or tetrachloro-3-6α-diphenylglycouril-3 attached to the antibody. Antibodies, particularly the monoclonal antibodies as described herein, may also be reacted with an enzyme in the presence of a coupling agent such as glutaraldehyde or periodate. Conjugates with fluorescein markers are conventionally prepared in the presence of these coupling agents or by reaction with an isothiocyanate. In some aspects, an anti-PD-1 antibody as described herein, particularly a binding fragment thereof, may be coupled or linked to a compound or substance, such as polyethylene glycol (PEG), to increase its in vivo half-life in plasma, serum, or blood following administration.

In some aspects, the antibodies described herein can be specifically bind to their intended target. In some aspects, the antibodies described herein have no off site binding.

### Methods

Disclosed herein are methods for treating an autoimmune disease in a subject. The methods can comprise administering to the subject a therapeutically effective amount of any of the isolated antibodies described herein or any of the compositions described herein. In some aspects, the autoimmune disease can be multiple sclerosis, type-1 diabetes, systemic lupus erythematosus, or rheumatoid arthritis. In some aspects, the autoimmune disease can be a T-cell mediated autoimmune disease. In some aspects, the autoimmune disease can be a PD-1 cell positive autoimmune disease.

Further disclosed herein are methods of treating or preventing a relapse associated with an autoimmune disease in a subject. In some aspects, the methods can comprise administering to the subject (e.g., a patient in remission for an autoimmune disease) a therapeutically effective amount of any of the isolated antibodies described herein or any of the composition disclosed herein. In some aspects, the subject can be in a remission stage of an autoimmune disease. The remission stage of the autoimmune disease can mean that the autoimmune disease is no longer active. In some aspects, the autoimmune disease can be multiple sclerosis, type-1 diabetes, systemic lupus erythematosus, or rheumatoid arthritis. In some aspects, the autoimmune disease can be a T-cell mediated autoimmune disease. In some aspects, the autoimmune disease can be a PD-1 cell positive autoimmune disease.

Also disclosed herein are methods of treating or reducing metastatic cancer in a subject or preventing metastasis in a subject having cancer at risk for metastasis. Also disclosed herein are methods of preventing reoccurrence of a tumor in a subject. In some aspects, the method comprises administering therapeutically effective amount of any of the isolated antibodies disclosed herein or any of the compositions disclosed herein. In some aspects, the cancer can be a cancer of breast, colon, lymphatic system, pancreas, lung, skin (including melanoma), esophagus, bladder, head and neck, or stomach. In some aspects, the subject has cancer. In some aspects, the subject has metastatic cancer. In some aspects, the subject can have cancer or be a cancer patient and is at risk for cancer metastasis. In some aspects, administration of any of the antibodies disclosed herein can reduce the number of metastases. In some aspects, administration of any of the antibodies disclosed herein can prevent the occurrence or reoccurrence of metastasis. In some aspects, administration of any of the antibodies disclosed herein can increase the subject's or patient's survival time. In some aspects, administration of any of the antibodies disclosed herein can prevent the reoccurrence of a tumor in the subject.

In some aspects, the subject can be identified in need of treatment before the administering step. In some aspects, the antibody can be administered in a pharmaceutically acceptable composition. In some aspects, the antibody can be administered systemically, intravenously, intradermally, intramuscularly, intraperitoneally, subcutaneously or locally into inflamed tissues, organs or tumors.

In some aspects, the methods can further comprising administering one or more drugs or therapeutic agents to the subject. Examples of drugs or therapeutic agents that can be administered in combination with any of the antibodies described herein, and in some aspects, to a subject with cancer, include but are not limited to chemotherapeutic agents, radiotherapy, immunotherapy, and surgery.

Examples of drugs or therapeutic agents that can be administered in combination with any of the antibodies described herein, and in some aspects, to a subject with an autoimmune disease or disorder, include but are not limited to but are not limited for type-1 diabetes, symptom relieving or management agents and insulin; and for multiple sclerosis, physical therapy, muscle relaxants, and medications to reduce fatigue.

Treatment of diseases. Disclosed herein are antibodies or antigen binding fragments thereof, as described herein (e.g., an antibody that specifically and preferentially binds to PD-1 and blocks or inhibits binding of PD-1 to PD-L1) that can be used in treatment methods and administered to treat or prevent an autoimmune disease or disorder, cancer, or metastatic cancer. Accordingly, provided herein are methods of treating an autoimmune disease, treating cancer, and treating metastatic cancer or preventing metastasis in a subject having cancer at risk for metastasis. In some aspects, the methods can comprise administering to a subject a therapeutically effective amount of any of the antibodies described herein or any of the compositions comprising at least one of antibodies as described herein. In some aspects, the drug or therapeutic agent can be an anti-PD-1 antibody or a composition comprising at least one anti-PD-1 antibody.

Also, disclosed herein are antibodies or antigen binding fragments thereof, as described herein (e.g., an antibody that specifically and preferentially binds to PD-1 and blocks or inhibits binding of PD-1 to PD-L1) that can be used in treatment methods and administered to deplete αPD-1 positive cells in a subject. In some aspects, the PD-1 positive cells can be malignant. In some aspects, the PD-1 positive cells can be lymphocytes. In some aspects, the PD-1 positive cells can be autoreactive immune cells. In some aspects, the method comprises removing PD-1 positive cells from the subject. In some aspects, the methods can comprise administering to a subject a therapeutically effective amount of any of the antibodies described herein or any of the compositions comprising at least one of antibodies as described herein. In some aspects, the drug or therapeutic agent can be an anti-PD-1 antibody or a composition comprising at least one anti-PD-1 antibody.

Further, disclosed herein are antibodies or antigen binding fragments thereof, as described herein (e.g., an antibody that specifically and preferentially binds to PD-1 and blocks or inhibits binding of PD-1 to PD-L1) that can be used in treatment methods and administered to prevent or treat lymphoma in a subject. In some aspects, the methods can comprise administering to a subject a therapeutically effective amount of any of the antibodies described herein or any of the compositions comprising at least one of antibodies as described herein. In some aspects, the drug or therapeutic agent can be an anti-PD-1 antibody or a composition comprising at least one anti-PD-1 antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with an autoimmune disease or cancer, lymphoma, or positive PD-1 positive cells in an amount sufficient to at least partially improve a sign or symptom or to inhibit the progression of (and preferably arrest) the symptoms of the disease or condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the autoimmune disease or cancer is delayed, hindered, or prevented, or the autoimmune disease or cancer or a symptom of the autoimmune disease or disorder is ameliorated or its frequency can be reduced. One or more of the symptoms can be less severe. Recovery can be accelerated in an individual who has been treated. For example, treatment of metastatic cancer may involve, for example, a reduction in the size of a tumor, a reduction in the invasiveness of a tumor, reduction in the growth rate of the cancer, or prevention of metastasis. Treatment of metastatic cancer may also refer to prolonging survival of a subject with cancer. In some aspects, the antibodies described herein can prolong the lifespan of a subject with cancer. In some aspects, the antibodies described herein can reduce or inhibit tumor cell growth.

Treatment of these subjects with an effective amount of at least one of the anti-PD-1 antibodies as described herein can result in binding of one or more of the disclosed antibodies to PD-1, thereby preventing, blocking or inhibiting PD-1 from binding to its cognate receptor, PD-L1, and thereby preventing or avoiding immune system (over) activity or activation of T-cells (or inducing suppression of T-cell activity). Accordingly, the methods as provided are advantageous for a subject who is in need of, capable of benefiting from, or who is desirous of receiving the benefit of, the anti-cancer results or the amelioration of one or more autoimmune symptoms, results achieved by the practice of the present methods. A subject's seeking the therapeutic benefits of the methods involving administration of at least one anti-PD-1 antibody in a therapeutically effective amount, or receiving such therapeutic benefits offer advantages to the art. In addition, the present methods offer the further advantages of eliminating or avoiding side effects, adverse outcomes, contraindications, and the like, or reducing the risk or potential for such issues to occur compared with other treatments and treatment modalities.

Autoimmune diseases for which the present methods are useful include but are not limited to multiple sclerosis, type-1 diabetes, systemic lupus erythematosus, or rheumatoid arthritis.

Cancers for which the present methods are useful include but are not limited to breast cancer, colon cancer, lymphatic system cancers, pancreatic cancer, lung cancer, skin cancers (including melanoma), esophageal cancer, bladder cancer, head and neck cancer and stomach cancer.

The anti-PD-1 antibodies, such as monoclonal antibodies, can be used as immunosuppressant agents in a variety of modalities. In some aspects, the methods described herein use the antibodies disclosed herein as immunosuppressant agents, and, thus, comprise contacting a population of cells with a therapeutically effective amount of one or more of the antibodies, or a composition containing one or more of the antibodies, for a time period sufficient to block or inhibit one or more of the following: T-cell chemotaxis. MHC-incompatible T-cell migration to lymph nodes, T-cell adherence to endothelium, T-cell migration to lymph nodes, intestinal mucosa, and skin, dendritic cell adherence to endothelium, dendritic cell migration to lymph nodes, and/or intestinal mucosa and skin. In some aspects, depletion of PD-1 positive cells can can cause acute immune surpression. In some aspects, contacting a cell in vivo is accomplished by administering to a subject in need, for example, by intravenous, subcutaneous, intraperitoneal, or intratumoral injection, a therapeutically effective amount of a physiologically tolerable composition comprising an anti-PD-1 antibody as described herein. The antibody may be administered parenterally by injection or by gradual infusion over time. Useful administration and delivery regimens include intravenous, intraperitoneal, oral, intramuscular, subcutaneous, intracavity, transdermal, dermal, peristaltic means, or direct injection into the tissue containing the cells.

Therapeutic compositions comprising antibodies are conventionally administered intravenously, such as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent. i.e., carrier, or vehicle. The compositions comprising any of the anti-PD-1 antibodies disclosed herein can be administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimens for initial and booster administration are also contemplated and may typically involve an initial administration followed by repeated doses at one or more intervals (hours) by a subsequent injection or other administration. In some aspects, multiple administrations can be suitable for maintaining continuously high serum and tissue levels of antibody. Alternatively, continuous intravenous infusion sufficient to maintain concentrations in the blood in the ranges specified for in vivo therapies are contemplated.

It is contemplated that an anti-PD-1 antibody as described herein can be administered systemically or locally to treat disease, such as to inhibit tumor cell growth or to kill cancer cells in cancer patients with locally advanced or metastatic cancers or at risk for metastatic cancers. The antibodies can be administered alone or in combination with anti-proliferative drugs or anticancer drugs. In some aspects, the anti-PD-1 antibodies can be administered to reduce the cancer load in the patient prior to surgery or other procedures. Alternatively, they can be administered at periodic intervals after surgery to ensure that any remaining cancer (e.g., cancer that the surgery failed to eliminate) is reduced in size or growth capacity and/or does not survive. As noted herein, a therapeutically effective amount of an antibody can be a predetermined amount calculated to achieve the desired effect. Thus, the dosage ranges for the administration of an anti-PD-1 antibody are those large enough to produce the desired effect in which the symptoms of tumor cell division and cell cycling are reduced. Optimally, the dosage should not be so large as to cause adverse side effects, such as hyperviscosity syndromes, pulmonary edema, congestive heart failure, neurological effects, and the like. Generally, the dosage will vary with age of, condition of, size and gender of, and extent of the disease in the subject or patient and can be determined by one of skill in the art such as a medical practitioner or clinician. Of course, the dosage may be adjusted by the individual physician in the event of any complication.

It is also contemplated that an anti-PD-1 antibody as described herein can be administered systemically or locally to treat an autoimmune disease, such as to block, inhibit or prevent PD-1 binding to PD-L1, and deplete PD-1 positive T cells, B cells, and other immune cells. The antibodies can be administered alone or in combination with other drugs or therapeutic agents.

Treatment methods. In some aspects, the compositions and methods as described herein comprise the administration of an anti-PD-1 antibody as described herein, alone, or in combination with a second or additional drug or therapy. Such drug or therapy may be applied in the treatment of any disease that is associated with PD-1, and in some aspects, the interaction of human PD-1 or with human PD-L1. For example, the disease can be an autoimmune disease, cancer, metastatic cancer, or any disease that comprises T-cells that are PD-1 positive. The compositions and methods described herein can comprise at least one anti-PD-1 antibody that preferentially binds to PD-L1 protein and has a therapeutic or protective effect in the treatment of an autoimmune disease, metastatic cancer or cancer, particularly by preventing, reducing, blocking, or inhibiting the PD-1/PD-L1 interaction, thereby providing a therapeutic effect and treatment.

The compositions and methods, including combination therapies, have a therapeutic or protective effect and may enhance the therapeutic or protective effect, and/or increase the therapeutic effect of another drug, therapy or therapeutic agent (e.g., anti-cancer or anti-hyperproliferative therapy).

Therapeutic and prophylactic methods and compositions can be provided in a combined amount effective to achieve the desired effect, such as the killing of a cancer cell and/or the inhibition of cellular hyperproliferation; reducing one or more symptoms of an autoimmune disease, reducing PD-1 positive cells). This process may involve administering an anti-PD-1 antibody or a binding fragment thereof and a second therapy. The second therapy may or may not have a direct cytotoxic effect. A tissue, tumor, and/or cell can be exposed to one or more compositions or pharmacological formulation(s) comprising one or more of the agents (e.g., an antibody or an anti-cancer agent), or by exposing the tissue, tumor, and/or cell with two or more distinct compositions or formulations, wherein one composition provides, for example, 1) an antibody, 2) an anti-cancer agent, 3) both an antibody and an anti-cancer agent, or 4) two or more antibodies. In some aspects, the second therapy can be also an anti-PD-1 antibody. Also, it is contemplated that such a combination therapy can be used in conjunction with chemotherapy, radiotherapy, surgical therapy, or immunotherapy.

By way of example, the terms “contacted” and “exposed,” when applied to a cell, are used herein to describe a process by which a therapeutic polypeptide, for example, an anti-PD-1 antibody as described herein, is delivered to a target cell or is placed in direct juxtaposition with the target cell, particularly to bind specifically to the target antigen, e.g., PD-1, expressed or highly expressed on the surface of endothelial venules, T-cell zones in lymph nodes and other secondary lymphoid organs. Such binding by a therapeutic anti-PD-1 antibody or binding fragment thereof prevents, blocks, inhibits, or reduces the interaction of PD-1 with PD-L1 on an effector T-cell, thereby preventing immune system activation associated with the PD-1/PD-L1 interaction. In some aspects, a chemotherapeutic or radiotherapeutic agent can also be administered or delivered to the subject in conjunction with the anti-PD-1 antibody or binding fragment thereof. To achieve cell killing, for example, one or more agents are delivered to a cell in a combined amount effective to kill the cell or prevent it from dividing.

Any of the anti-PD-1 antibodies disclosed herein may be administered before, during, after, or in various combinations relative to another treatment (e.g., anti-cancer, or immunosuppressant agent). The administrations may be in intervals ranging from concurrently to minutes to days to weeks before or after one another. In some aspects, in which the antibody is provided to a patient separately from an anti-cancer agent or immunosuppressant agent, it would be generally ensured that a significant period of time did not expire between the time of each delivery, such that the administered compounds would still be able to exert an advantageously combined effect for the patient. Illustratively, in such instances, it is contemplated that one may provide a patient with the antibody and the anti-cancer therapy or immunosuppressant agent within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment or treatment cycle will last 1-90 days or more (this range includes intervening days and the last day). It is contemplated that one agent may be given on any day of day 1 to day 90 (this such range includes intervening days and the last day) or any combination thereof, and another agent is given on any day of day 1 to day 90 (this such range includes intervening days and the last day) or any combination thereof. Within a single day (24-hour period), the patient may be given one or multiple administrations of the agent(s). Moreover, after a course of treatment, it is contemplated that there may be a period of time at which no second agent (e.g., anti-cancer treatment or immunosuppressant agent) is administered. This time period may last, for example, for 1-7 days, and/or 1-5 weeks, and/or 1-12 months or more (this such range includes intervening days and the upper time point), depending on the condition of the patient, such as prognosis, strength, health, etc. Treatment cycles would be repeated as necessary. Various combinations of treatments may be employed. In the representative examples of combination treatment regimens shown below, an antibody, such as an anti-PD-1 antibody or binding fragment thereof is represented by “A” and an anti-cancer therapy is represented by “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

Administration of any antibody or therapy as described herein to a patient will follow general protocols for the administration of such compounds, taking into account the toxicity, if any, of the agents. Therefore, in some aspects there is a step of monitoring adverse events and toxicity, particularly those that may be attributable to combination therapy.

In some aspects, methods are disclosed comprising administering an anti PD-1 antibody alone or in combination with another agent (e.g., anticancer agent or immunosuppressant agent) to a subject in need thereof, i.e., a subject with a cancer or a tumor, an autoimmune disease, or PD-1 positive cells). Prior to administration of the anti-PD-1 antibody, a sample of the subject's tumor or cancer or one or more symptoms associated with the autoimmune disease may be evaluated for the presence or level of PD-1. If the results of such an evaluation reveals that the subject's tumor or cancer or symptoms associated with the autoimmune disease is positive for PD-1 or the level of PD-1 or PD-1 positive cells is increased compared to a reference sample or prior sample from the same subject, the subject would be selected for treatment based on the likelihood that subject's PD-1⁺ tumor or cancer or disease state or condition would be more amenable to treatment with the anti-PD-1 antibody and treatment may proceed with a more likely beneficial outcome. A medical professional or physician may advise the subject to proceed with the anti-PD-1 antibody treatment method, and the subject may decide to proceed with treatment based on the advice of the medical professional or physician. In addition, during the course of treatment, the subject's tumor or cancer cells or blood cells may be assayed for the presence of PD-1 as a way to monitor the progress or effectiveness of treatment. If the assay shows a change, loss, or decrease, for example, in PD-1 on the subject's tumor or cancer cells or blood cells, a decision may be taken by the medical professional in conjunction with the subject as to whether the treatment should continue or be altered in some fashion, e.g., a higher dosage, the addition of another anti-cancer agent or therapy or immunosuppressant, and the like.

Chemotherapy. A wide variety of chemotherapeutic agents may be used in accordance with the treatment or therapeutic methods as described herein. The term “chemotherapy” refers to the use of drugs to treat cancer. A “chemotherapeutic agent” connotes a compound or composition that is administered in the treatment of cancer. Such agents or drugs are categorized by their mode of activity within a cell, for example, whether and at what stage they affect the cell cycle and cell growth and proliferation. Alternatively, a chemotherapeutic agent may be characterized based on its ability to directly cross-link DNA, to intercalate into DNA, or to induce chromosomal and mitotic aberrations by affecting nucleic acid synthesis in a cell.

Nonlimiting examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide: mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine, plicomycin, gemcitabine, navelbine, farnesyl-protein tansferase inhibitors, transplatinum, and pharmaceutically acceptable salts, acids, or derivatives of any of the above.

Radiotherapy. Radiotherapy includes treatments with agents that cause DNA damage. Radiotherapy has been used extensively in cancer and disease treatments and embraces what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA itself, on the precursors of DNA, on the replication and repair of DNA, and on the assembly and maintenance of chromosomes. Exemplary dosage ranges for X-rays range from daily doses of 50 to 200 roentgens for prolonged periods of time (3 to 4 weeks) to single doses of 2000 to 6000 roentgens. Dosage ranges for radioisotopes vary widely and depend on the half-life of the isotope, the strength and type of radiation emitted, the uptake by the neoplastic cells, and tolerance of the subject undergoing treatment.

Immunotherapy. In some aspects of the methods, the anti-PD-1 antibodies as described herein can be administered after patients are found to be resistent to one or more of the following immunotherapies including but not limited to Rituximab (RITUXAN®), checkpoint inhibitors including ipilimumab, anti-PD-1 or anti-PD-L1 inhibitors, such as antibodies against PD-L1, which include atezolizumab, durvalumab, or avelumab, or antibodies against PD-1, including nivolumab, pembrolizumab, or pidilizumab. Any of these therapeutics can lead to an increased number of PD-1 positive tumor cells, and the antibody disclosed herein can solve the resistance by binding to the PD-1 positive tumor cells, and deplete them.

In some aspects, the antibody disclosed herien also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target, e.g., the PD-1 on T-cells/PD-L1 on tumor cells interaction. Various effector cells include cytotoxic T cells and natural killer (NK) cells.

In the context of treating an autoimmune disease or disorder, one or more of the immunotherapeutics can be administered to the subject.

In some aspects of immunotherapy, the tumor cell must bear some marker (protein/receptor) that is amenable to targeting. Optimally, the tumor marker protein/receptor is not present on the majority of other cells, such as non-cancer cells or normal cells. Many tumor markers exist and any of these may be suitable for targeting by another drug or therapy administered with an anti-PD-1 antibody as disclosed herein. Common tumor markers include, for example CD20, carcinoembryonic antigen (CEA), tyrosinase (p97), gp68, TAG-72, HMFG. Sialyl Lewis Antigen, MucA, MucB, PLAP, laminin receptor, erbB, and p155. An alternative aspect of immunotherapy is to combine anticancer effects with immune stimulatory effects. Immune stimulating molecules also exist and include cytokines, such as IL-2, IL-4, IL-12, GM-CSF, gamma-IFN; chemokines, such as MIP-1, MCP-1, IL-8; and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui et al., 1998, Infection Immun., 66(11); 5329-5336; Christodoulides et al., 1998, Microbiology, 144 (Pt 11); 3027-3037); cytokine therapy, e.g., α, β, and γ interferons; IL-1, GM-CSF, and TNF (Bukowski et al., 1998, Clinical Cancer Res., 4(10); 2337-2347; Davidson et al., 1998, J. Immunother., 21(5); 389-398; Hellstrand et al., 1998, Acta Oncologica, 37(4); 347-353); gene therapy, e.g., TNF, IL-1, IL-2, and p53 (Qin et al., 1998, Proc. Natl. Acad. Sci. USA, 95(24); 14411-14416; Austin-Ward and Villaseca, 1998. Revista Medica de Chile, 126(7); 838-845; U.S. Pat. Nos. 5,830,880 and 5,846,945); and monoclonal antibodies, e.g., anti-CD20, anti-ganglioside GM2, and anti-p185 (Hollander, 2012. Front. Immun., 3:3; Hanibuchi et al., 1998, Int. J. Cancer, 78(4); 480-485; U.S. Pat. No. 5,824,311). It is contemplated that one or more anti-cancer therapies may be employed with the antibody therapies described herein.

Surgery. Approximately 60% of individuals with cancer undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as anti-PD-1 antibody treatment as described herein, chemotherapy, radiotherapy, hormonal therapy, gene therapy, immunotherapy, and/or alternative therapies, as well as combinations thereof. Tumor resection refers to physical removal of at least part of a tumor. In addition to tumor resection, treatment by surgery includes laser surgery, cryosurgery, electrosurgery, and microscopically-controlled surgery (Mohs' surgery). Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1. 2, 3, 4, 5, 6, 7, 8, 9, 10, 11, or 12 months. These treatments may be of varying dosages as well.

Protein Purification. Protein, including antibody and, particularly, anti-PD-1 antibody, purification techniques are well known to those of skill in the art. These techniques involve, at one level, the homogenization and crude fractionation of the cells, tissue, or organ into polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure protein or peptide are ion-exchange chromatography, size-exclusion chromatography, reverse phase chromatography, hydroxyapatite chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC). As is generally known in the art, the order of conducting the various purification steps may be changed, and/or certain steps may be omitted, and still result in a suitable method for the preparation of a substantially purified polypeptide.

A purified polypeptide, such as an anti-PD-1 antibody as described herein, refers to a polypeptide which is isolatable or isolated from other components and purified to any degree relative to its naturally-obtainable state. An isolated or purified polypeptide, therefore, also refers to a polypeptide free from the environment in which it may naturally occur, e.g., cells, tissues, organs, biological samples, and the like. Generally. “purified” will refer to a polypeptide composition that has been subjected to fractionation to remove various other components, and which composition substantially retains its expressed biological activity. A “substantially purified” composition refers to one in which the polypeptide forms the major component of the composition, and as such, constitutes about 50%, about 60%, about 70%, about 80%, about 90%, about 95%, or more of the protein component of the composition.

Various methods for quantifying the degree of purification of polypeptides, such as antibody proteins, are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed polypeptide exhibits a detectable activity.

There is no general requirement that the polypeptide will always be provided in its most purified state. Indeed, it is contemplated that less substantially purified products may have utility in some aspects. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance (protein) to be isolated and a molecule to which it can specifically bind, e.g., a receptor-ligand type of interaction. The column material (resin) is synthesized by covalently coupling one of the binding partners to an insoluble matrix. The column material is then able to specifically adsorb the substance from the solution that is passed over the column resin. Elution occurs by changing the conditions to those in which binding will be disrupted/will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding; however, elution of the bound substance should occur without destroying the sample protein desired or the ligand.

Size-exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase. The underlying principle of SEC is that particles of different sizes will elute (filter) through a stationary phase at different rates, resulting in the separation of a solution of particles based on size. Provided that all of the particles are loaded simultaneously or near simultaneously, particles of the same size should elute together.

High-performance (aka high-pressure) liquid chromatography (HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used

Pharmaceutical Preparations. Where clinical application of a pharmaceutical composition comprising an anti-PD-1 antibody is undertaken, it is generally beneficial to prepare a pharmaceutical or therapeutic composition appropriate for the intended application. In general, pharmaceutical compositions can comprise an effective amount of one or more polypeptides or additional agents dissolved or dispersed in a pharmaceutically acceptable carrier. In some aspects, pharmaceutical compositions may comprise, for example, at least about 0.1% of a polypeptide or antibody. In some aspects, a polypeptide or antibody may comprise between about 2% to about 75% of the weight of the unit, or between about 25% to about 60%, for example, and any range derivable there between, including the upper and lower values. The amount of active compound(s) in each therapeutically useful composition may be prepared in such a way that a suitable dosage will be obtained in any given unit dose. Factors, such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

Further in some aspects, the composition suitable for administration can be provided in a pharmaceutically acceptable carrier with or without an inert diluent. The carrier should be assimilable and include liquid, semi-solid, e.g., gels or pastes, or solid carriers. Examples of carriers or diluents include but are not limited to fats, oils, water, saline solutions, lipids, liposomes, resins, binders, fillers, and the like, or combinations thereof. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc.), gels, binders, excipients, disintegration agents, lubricants, sweetening agents, flavoring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in an administrable composition for the practice of the methods is appropriate. The pH and exact concentration of the various components in a pharmaceutical composition are adjusted according to well-known parameters. In some aspects, the composition can be combined with the carrier in any convenient and practical manner, i.e., by solution, suspension, emulsification, admixture, encapsulation, absorption, grinding, and the like. Such procedures are routine for those skilled in the art.

In some aspects, the compositions may comprise different types of carriers depending on whether they are to be administered in solid, liquid, or aerosol form, and whether it needs to be sterile for the route of administration, such as injection. The compositions can be formulated for administration intravenously, intradermally, transdermally, intrathecally, intra-arterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art. See, for example, Remington's Pharmaceutical Sciences, 18th Ed., 1990. Typically, such compositions can be prepared as either liquid solutions or suspensions; solid or reconstitutable forms suitable for use to prepare solutions or suspensions upon the addition of a liquid prior to injection can also be prepared; and, the preparations can also be emulsified.

The antibodies may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups may also be derived from inorganic bases, such as, for example, sodium, potassium, ammonium, calcium, or ferric hydroxides; or such organic bases as isopropylamine, trimethylamine, histidine, or procaine.

In some aspects, a pharmaceutical lipid vehicle composition that includes polypeptides, one or more lipids, and an aqueous solvent may be used. As used herein, the term “lipid” refers to any of a broad range of substances that are characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods. One of ordinary skill in the art would be familiar with the range of techniques that can be employed for dispersing a composition in a lipid vehicle. For example, the antibody may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic antibody or composition containing the therapeutic antibody calculated to produce the desired responses discussed above in association with its administration. i.e., the appropriate route and treatment regimen. The quantity to be administered, both according to number of treatments and unit dose, depends on the effect desired. The actual dosage amount of a composition as described herein that can be administered to a patient or subject can be determined by physical and physiological factors, such as body weight, the age, health, and sex of the subject, the type of disease being treated, the extent of disease penetration, previous or concurrent therapeutic interventions, idiopathy of the subject, the route of administration, and the potency, stability, and toxicity of the particular therapeutic substance. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above. The foregoing doses include amounts between those indicated and are intended to also include the lower and upper values of the ranges. The practitioner responsible for administration will, in any event, determine the concentration of active ingredient(s) in a composition and appropriate dose(s) for the individual subject.

The particular nature of the therapeutic composition or preparation is not intended to be limiting. For example, suitable compositions may be provided in formulations together with physiologically tolerable liquid, gel, or solid carriers, diluents, and excipients. In some aspects, the therapeutic preparations may be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects, as described supra.

PD-1 as a biomarker. Disclosed herein are methods comprising the use of at least one anti-CCL21 antibody as described herein. In some aspects, the methods can comprise detecting the amount of level of PD-1 gene or protein in a sample obtained from a subject who has a cancer or tumor or is exhibiting one or more symptoms of an autoimmune disease or disorder. Such methods may be useful in biomarker evaluations of the level of PD-1 in a sample obtained from a subject who has a cancer or tumor or is exhibiting one or more symptoms of an autoimmune disease or disorder. In some aspects, the autoimmune disease or disorder is multiple sclerosis, type-1 diabetes, systemic lupus erythematosus, or rheumatoid arthritis. For example, if the subject's sample is tested and determined to comprise a higher level of PD-1 or PD-1 positive cells compared to a reference sample, then the subject is a candidate for treatment with an anti-PD-1 antibody as described herein, alone, or in combination with another agent, for example, would benefit from the treatment. Such methods comprise obtaining a sample from a subject having a cancer or tumor (or exhibiting one or more symptoms of an autoimmune disease or disorder), testing the sample for the presence of PD-1 derived from the subject's sample using binding methods known and used in the art and as described herein, for example, using an anti-PD-1 antibody as described herein, and administering to the subject an effective amount of an anti-PD-1 antibody alone, or in combination with another agent, if the subject's sample is found to have a higher level of PD-1 when compared to a reference sample. Diagnosing the subject as having a cancer or tumor or an autoimmune disease prior to treatment allows for more effective treatment and benefit to the subject, as the administered anti-PD-1 antibody is more likely to block or inhibit the interaction of the subject's PD-1 with the subject's PD-11, thereby inducing immunosuppression of the T-cell activity or reducing T-cell activation or blocking T-cell migration or adherence. In some aspects, the methods can involve first selecting a subject whose cancer or tumor or disease state or condition may be amenable to testing for the presence of PD-1 levels.

Similar methods may be used to monitor the presence of PD-1 levels during a course of treatment or therapy, including combination treatments with an anti-PD-1 antibody and another anticancer drug or treatment or another immunosuppressant, over time, as well as after treatment has ceased. Such methods may also be used in companion diagnostic methods in which a treatment regimen or combination treatment, involves testing or assaying a sample obtained from the subject for PD-1 levels, prior to treatment and during the course of treatment, e.g., monitoring, to determine a successful outcome or the likelihood thereof.

Other Agents. It is contemplated that other agents may be used in combination with certain aspects of the compositions and methods disclosed herein to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions may increase the anti-hyperproliferative effects on the neighboring hyperproliferative cell population. In some aspects, cytostatic or differentiation agents may be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### Fusions and Conjugates

The anti-PD-1 antibodies or polypeptides disclosed herein can also be expressed as fusion proteins with other proteins or chemically conjugated to another moiety. In some aspects, the antibodies or polypeptides can have an Fc portion that can be varied by isotype or subclass, can be a chimeric or hybrid, and/or can be modified, for example to improve effector functions, control half-life or tissue accessibility, augment biophysical characteristics, such as stability, and improve efficiency of production, which can be associated with cost reductions. Many modifications useful in the construction of fusion proteins and methods for making them are known in the art, for example, as reported by Mueller. J. P. et al., 1997. Mol. Immun. 34(6); 441-452; Swann. P. G., 2008. Curr. Opin. Immunol., 20:493-499; and Presta, L. G., 2008, Curr. Opin. Immunol., 20:460-470. In some aspects, the Fc region can be the native IgG1, IgG2, or IgG4 Fc region of the antibody. In some aspects, the Fc region can be a hybrid, for example, a chimera containing IgG2/IgG4 Fc constant regions. Modifications to the Fc region include, but are not limited to. IgG4 modified to prevent binding to Fc gamma receptors and complement; IgG1 modified to improve binding to one or more Fc gamma receptors; IgG1 modified to minimize effector function (amino acid changes); and IgG1 with altered pH-dependent binding to FcRn. The Fc region can include the entire hinge region, or less than the entire hinge region of the antibody.

In some aspects, IgG2-4 hybrids and IgG4 mutants have reduced binding to FcR which can increase their half-life. Representative IG2-4 hybrids and IgG4 mutants are described, for example, in Angal et al., 1993, Molec. Immunol . . . 30(1); 105-10⁸; Mueller et al., 1997, Mol. Immun., 34(6); 441-452; and U.S. Pat. No. 6,982,323; all of which are hereby incorporated by references in their entireties. In some aspects, the IgG1 and/or IgG2 domain can be deleted. For example, Angal et al., Id., describe proteins in which IgG1 and IgG2 domains have serine 241 replaced with a proline. In some aspects, fusion proteins or polypeptides having at least 10, at least 20, at least 30, at least 40, at least 50, at least 60, at least 70, at least 80, at least 90 or at least 100 amino acids are contemplated.

In some aspects, anti-PD-1 antibodies or polypeptides can be linked to or covalently bind or form a complex with at least one moiety. Such a moiety may be, but is not limited to, one that increases the efficacy of the antibody as a diagnostic or a therapeutic agent. In some aspects, the moiety can be an imaging agent, a toxin, a therapeutic enzyme, an antibiotic, a radio-labeled nucleotide, a chemotherapeutic agent, and the like.

In some aspects, antibodies and polypeptides as described herein may be conjugated to a marker, such as a peptide, to facilitate purification. In some aspects, the marker can be a hexa-histidine peptide, i.e., the hemagglutinin “HA” tag, which corresponds to an epitope derived from the influenza hemagglutinin protein (Wilson, I. A. et al., Cell. 37:767-778 (1984)), or the “flag” tag (Knappik, A. et al., Biotechniques 17(4); 754-761(1994)).

In some aspects, the moiety conjugated to the antibodies and polypeptides as described herein can be an imaging agent that can be detected in an assay. Such imaging agents can be enzymes, prosthetic groups, radiolabels, nonradioactive paramagnetic metal ions, haptens, fluorescent labels, phosphorescent molecules, chemiluminescent molecules, chromophores, luminescent molecules, bioluminescent molecules, photoaffinity molecules, or colored particles or ligands, such as biotin. In some aspects, suitable enzymes include, but are not limited to, horseradish peroxidase, alkaline phosphatase, beta-galactosidase, or acetylcholinesterase; prosthetic group complexes include, but are not limited to, streptavidin/biotin and avidin/biotin; fluorescent materials include, but are not limited to, umbelliferone, fluorescein, fluorescein isothiocyanate, rhodamine, dichlorotriazinylamine fluorescein, dansylchloride or phycoerythrin; luminescent materials include, but are not limited to, luminol; bioluminescent materials include, but are not limited to, luciferase, luciferin, and aequorin; radioactive materials include, but are not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The imaging agent can be conjugated to the antibodies or polypeptides described herein either directly or indirectly through an intermediate (such as, for example, a linker known in the art) using techniques known in the art. See, for example, U.S. Pat. No. 4,741,900 which reports on metal ions that can be conjugated to antibodies and other molecules as described herein for use as diagnostics. Some conjugation methods involve the use of a metal chelate complex employing, for example, an organic chelating agent, such as diethylenetriaminepentaacetic acid anhydride (DTPA); ethylenetriaminetetraacetic acid; N-chloro-p-toluenesulfonamide; and/or tetrachloro-3-6α-diphenylglycouril-3, attached to the antibody. Monoclonal antibodies can also be reacted with an enzyme in the presence of a coupling agent such as glutaraldehyde or periodate. Conjugates with fluorescein markers can be prepared in the presence of these coupling agents or by reaction with an isothiocyanate.

In some aspects, the anti-PD-1 antibodies polypeptides as described herein can be conjugated to a second antibody to form an antibody heteroconjugate, for example, as described in U.S. Pat. No. 4,676,980. Such heteroconjugate antibodies can additionally bind to haptens (e.g., fluorescein), or to cellular markers.

In some aspects, the anti-PD-1 antibodies or polypeptides described herein can also be attached to solid supports, which can be useful for carrying out immunoassays or purification of the target antigen or of other molecules that are capable of binding to the target antigen that has been immobilized to the support via binding to an antibody or antigen binding fragment as described herein. Such solid supports include, but are not limited to, glass, cellulose, polyacrylamide, nylon, polystyrene, polyvinyl chloride or polypropylene.

### Kits and Diagnostics

Disclosed herein are kits comprising therapeutic agents and/or other therapeutic and delivery agents. In some aspects, the kits can be used for preparing and/or administering a therapy involving the anti-PD-1 antibodies described herein. The kits can comprise one or more sealed vials containing any of the pharmaceutical compositions as described herein. The kits can include, for example, at least one anti-PD-1 antibody, as well as reagents to prepare, formulate, and/or administer one or more anti-PD-1 antibodies or to perform one or more steps of the described methods. In some aspects, the kits can also comprise a suitable container means, which is a container that will not react with components of the kit, such as an Eppendorf tube, an assay plate, a syringe, a bottle, or a tube. The container may be made from sterilizable materials, such as plastic or glass.

The kits can further include an instruction sheet that outlines the procedural steps of the methods set forth herein, and will follow substantially the same procedures as described herein or are known to those of ordinary skill. The instruction information may be in a computer readable medium containing machine-readable instructions that, when executed using a computer, cause the display of a real or virtual procedure of delivering a pharmaceutically effective amount of the therapeutic agent.

### EXAMPLES

### Example 1: Antibody-Mediated Depletion of Programmed Death-1 Positive (PD-1+) Cells

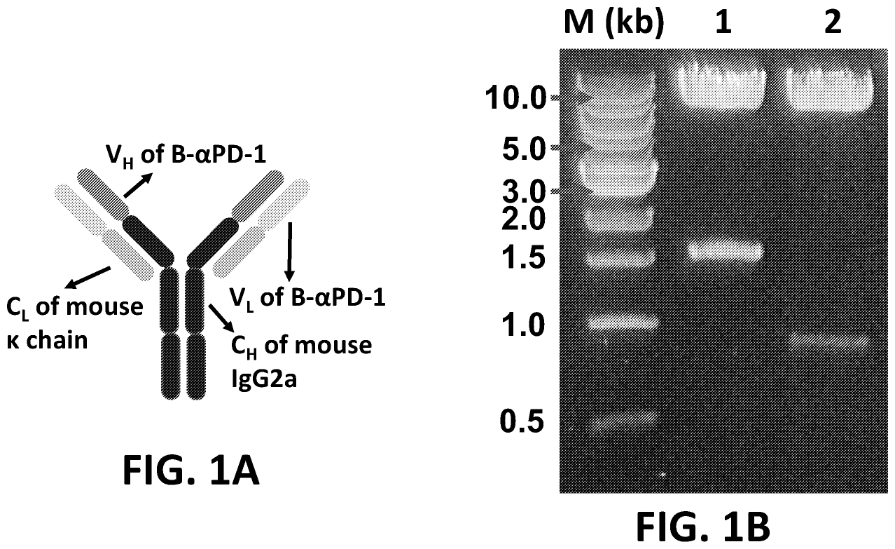

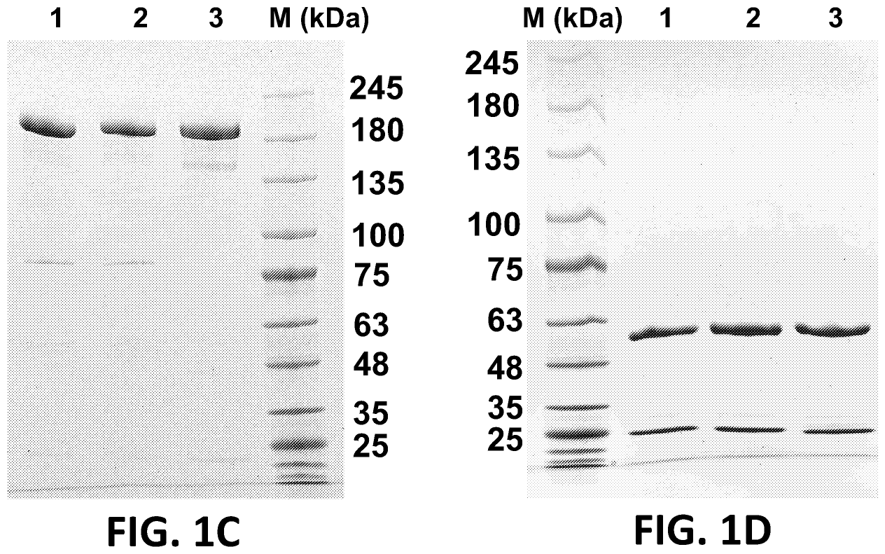

Results. Design and generation of a mouse depleting anti-PD-1 antibody (D-αPD-1). D-αPD-1 was produced as two recombinant proteins, a heavy chain and a light chain through a mammalian expression system. Then, the heavy chain and the light chain self-assembled into D-αPD-1 inside mammalian host cells. The coding gene for D-αPD-1 heavy chain was generated by fusing two genes together: one gene is for the heavy chain variable domain (V_{H}) of B-αPD-1 (RMP1-14) (Zhao, P., et al., Nat Biomed Eng, 2019. 3(4); p. 292-305); another gene is for three heavy chain constant domains of a mouse IgG2a (Genbank ID: BAC44883.1). The coding gene of D-αPD-1 light chain was generated by fusing two genes as well: one gene is for the light chain variable domain (V_{L}) of B-αPD-1; another gene is for the constant domain of a mouse κ chain (GenBank: BAB33404.1) (FIG. 1A). The coding genes of D-αPD-1 heavy and light chains were chemically synthesized and inserted in a pcDNA3.4 expression vector, respectively (FIG. 1A). The sizes of inserted coding genes were examined through enzymatic cleavage in combination with gel electrophoresis. The migration positions of inserted genes for D-αPD-1 light and heavy chains on the gel suggest that the sizes of these two genes are approximately 700 bp and 1500 bp, respectively. These estimated sizes are consistent with the theoretical sizes of the coding genes (light chain 723 bp and heavy chain 1413 bp, (FIG. 1B)). The sequence of coding genes on the expression vectors were confirmed by DNA sequencing.

To express D-αPD-1, the expression vectors harboring encoding genes of light and heavy chains were used to co-transfect Expi293F cells at the 2:1 ratio as suggested by the protocol of the expression system. The assembled D-αPD-1 was secreted from Expi293F cells. D-αPD-1 was purified using protein G beads. On a non-reducing SDS-PAGE gel, D-αPD-1 appear to have a similar MW to B-αPD-1 and an IgG2a isotype control. On a reduced SDS gel, D-αPD-1 shows in two bands: a higher band of approximately 50 kDa represents heavy chains and a lower band of approximately 25 kDa represents light chains (FIG. 1D).

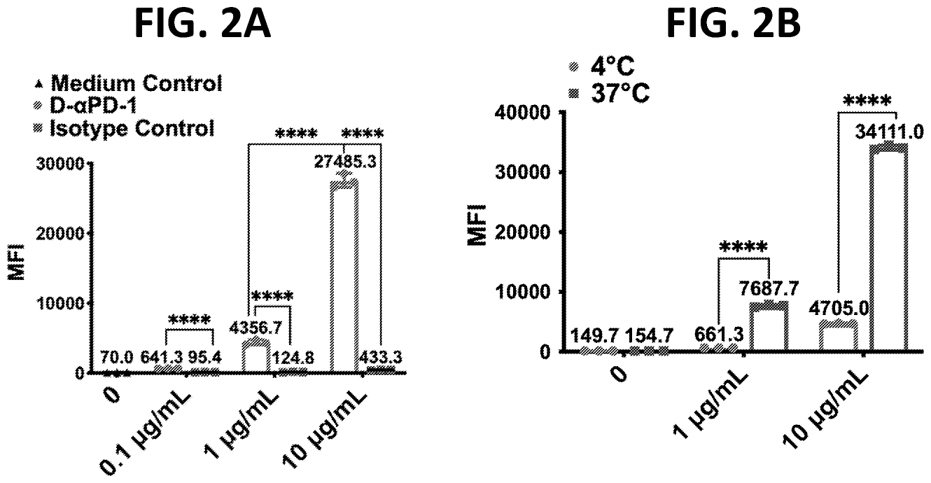

D-αPD-1 selectively binds to PD-1, cells. The interactions between D-αPD-1 and cells at 4° C., a condition that allows for the binding between an antibody and its cell surface antigens but inhibits the internalization of the antigens, was studied. Here, D-αPD-1 and an IgG2a isotype control at three different concentrations were incubated with EL4 cells that are PD-1⁺. Then, bound antibodies were stained with PE-labeled, anti-mouse IgG2a antibody and measured by flow cytometry. The mean MFI resulted from the incubation between EL4 cells and the isotype control is statistically significantly lower than that from the incubation between EL4 cells and D-αPD-1 at all three concentration settings (P<0.0001 FIG. 2A). Further, the binding between D-αPD-1 and EL4 cells was found to be dose-dependent (FIG. 2A). For the three used concentrations, 0.1, 1.0, 10.0 μg/mL, the higher the D-αPD-1 concentration, the greater the mean MFI. Specifically, when the concentration of D-αPD-1 increased from 1 μg/mL to 10 μg/mL, the mean of MFI raised by approximately 6 times (4356.7 vs 27485.3, P<0.0001). In contrast, the increased concentrations of the isotope control did not result in an increase of MFI. Together, these results show that D-αPD-1 is able to bind with PD-1⁺ cells, which is the basis for the depletive effect of D-αPD-1.

Next, the interactions between D-αPD-1 and cells were compared at 4° C. and 37° C. The latter condition allows the binding of antibodies with their cell surface antigens as well as the internalization of the antigens. To conduct the comparison, Alexa Fluor 647-labeled D-αPD-1 was incubated with EL4 cells at two different concentrations (1 and 10 μg/mL) and two different temperatures. After that, the cells were analyzed by flow cytometry. When no D-αPD-1 is used for incubation, EL4 cells have low and similar MFIs at both temperatures. When 1 μg/mL D-αPD-1 was used for the incubation, the mean MFL at 37° C. is approximately 10 times higher than that at 4° C. (7687.7 vs 661.3, P<0.0001). This trend is maintained when D-αPD-1 was used at 10 μg/mL. The mean MFI resulted from the 37° C. incubations is more than 7-time greater than the mean MFI from the 4° C. incubations (34111.0 vs 4705.0, P<0.0001). These data clearly showed that at 37° C., a greater amount of D-αPD-1 is associated with EL4 cells, which may be caused by internalization of PD-1.

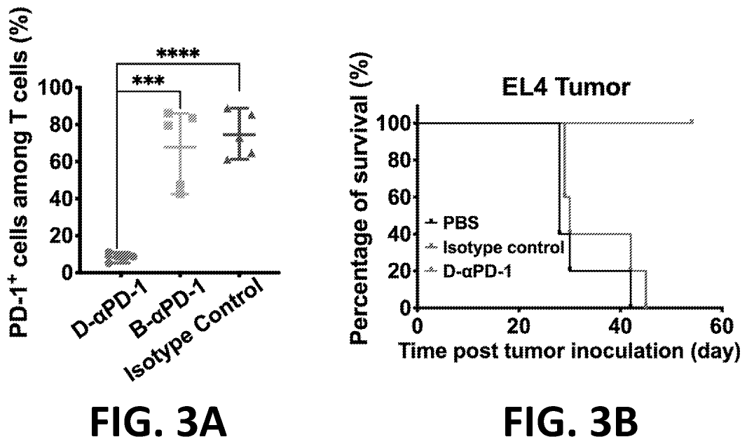

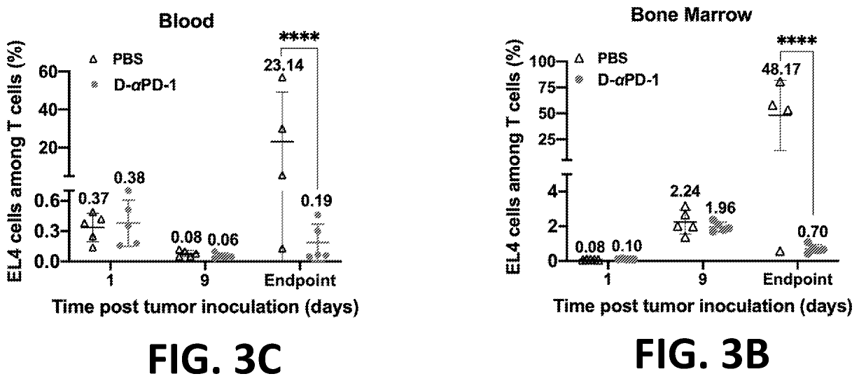

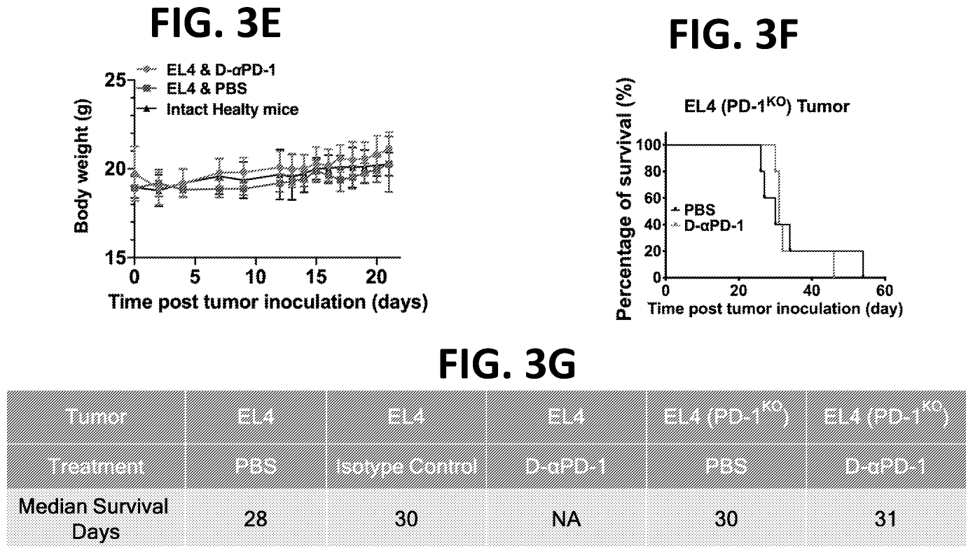

The treatment of D-αPD-1 leads to elimination of PD-1⁺ cells in vivo. Because the antibody-mediated cell depletion requires the involvement of a range of immune effector cells and molecules, it is difficult to reproduce complex conditions and assess antibody-mediated depletion in vitro. Thus, an in vivo depletion assay was designed to assess the effectiveness of D-αPD-1 to deplete PD-1⁺ cells. Here, EL4 cells were transferred into mice to boost numbers of PD-1⁺ cells in mouse bodies. Then, the mice were treated with D-αPD-1, B-αPD-1, or the IgG2a isotype control at day 1 and day 10 after cell transfer. At 12 days after cell transfer, PD-1⁺ cells were quantified in bone marrows of transferred mice, where a large number of PD-1⁺ cells accumulated. The results show that the treatment of D-αPD-1 significantly reduced PD-1⁺ cells. Among D-αPD-1 treated mice, the mean fraction of PD-1⁺ cells among T cells is 8%. This mean is significantly lower than the means of B-αPD-1 and the isotype control-treated mice, at 68% and 75%, respectively (P<0.001 and P<0.0001) (FIG. 3A). This result indicates that D-αPD-1 has the capability to specifically eliminate PD-1⁺ cells in vivo. Compared to D-αPD-1, B-αPD-1 has the same antigen-binding sites for mouse PD-1 but lacks an IgG2a Fc. Thus, while B-αPD-1 can bind with mouse PD-1⁺ cells, it should not be able to initiate antibody-mediated elimination of these cells. On the other hand, compared to D-αPD-1, the isotype control has IgG2a Fc but not antigen-binding sites for mouse PD-1. Thus, the control should not eliminate PD-1⁺ cells. The above result confirmed these expectations.

EL4 is a tumor line syngeneic to C57B/L6 mice. EL4 cells inoculated into this strain of mice, if not eliminated, can grow into tumors that are lethal to the mice. Thus, survival of EL4-inoculated mice was used as an additional measurement of the depleting effect of D-αPD-1. Here, mice were inoculated with EL4 cells at day 0 and treated with D-αPD-1, IgG2a isotype control, or PBS at day 1. The median survival time for the PBS treated mice and the isotype control treated mice were 28 and 30 days after tumor inoculation, respectively (FIGS. 3B and 3G). Further, no mice in these two treatment groups survived beyond 49 days. In contrast, the D-αPD-1 treated mice are still alive at 80 days after tumor inoculation, when this study was terminated (FIGS. 3B and 3G). The survival of D-αPD-1 treated mice is significantly longer than PBS and the isotype control treated mice (P<0.001). The 100% survival of the D-αPD-1 treated mice verifies the potent depleting effect of D-αPD-1 on PD-1⁺ cells. With the same design of EL4 cell transfer, the presence of EL4 cells in mice that received PBS and D-αPD-1 treatment, respectively, were compared. The cells were examined at three time points, 1 and 9 days after cell transfer, and the humane endpoint for mice in the PBS treated group. Mice in the D-αPD-1 treated group were euthanized and examined at the time matching the endpoints of PBS-treated mice. The results show EL4 cells are present at very low frequencies in blood and bone marrows of both treatment groups (FIGS. 3C and 3D). However, at the endpoints, while D-αPD-1 treated group showed a marginal amount of EL4 cells in blood and bone marrows, PBS treated mice have explosive numbers of EL4 cells. The average EL4 cell fractions among CD3 cells are 0.19% and 0.70% for blood and bone marrow samples of D-αPD-1 treated mice. These averages are significantly lower than those values of PBS-treated mice, 23.12% and 48.17% (P<0.0001 for both comparisons). These results further reinforced that D-αPD-1 treatment eliminated EL4 cells in mice by targeting PD-1⁺ cells.

The results also show that the D-αPD-1 treatment was tolerated from the toxicity perspective, evidenced by body weight data of three treatment groups: intact mice, mice receiving EL4 cell transfer and the PBS treatment, and mice receiving EL4 cell transfer and the D-αPD-1 treatment (FIG. 3E). The body weights of these mice were measured up to 21 days after the PBS and D-αPD-1 treatments. The mice that received the D-αPD-1 treatment maintained the same body weight growth trend as the other two groups including intact healthy mice, indicating that the D-αPD-1 antibody does not cause any severe side effect that amounts to a body weight loss.

It was further examined whether the depleting effect of D-αPD-1 depends on its binding with PD-1 on PD-1⁺ cells. For the examination, mice were inoculated with EL4 (PD-1^{KO}) cells (Zhao. P., et al., Nat Biomed Eng. 2019. 3(4); p. 292-305) and treated with either D-αPD-1 or PBS. In a sharp contrast to mice inoculated with EL4 cells, mice inoculated with EL4 (PD-1^{KO}) cells did not respond to the treatment of D-αPD-1. The survival time of the D-αPD-1 treated group and the PBS treated group are not statistically different (P=0.98, FIG. 3F). The mean survival days were 30 and 31 days post tumor inoculation, respectively (FIG. 3G). The mice in the D-αPD-1 treated group had to be euthanized by 6 weeks post tumor inoculation due to the growth of EL4 tumors. This result shows D-αPD-1 has no therapeutic effect on EL4 (PD-1^{KO}) tumors, suggesting D-αPD-1 relies on PD-1 on PD-1⁺ cells to induce ablation of these cells.

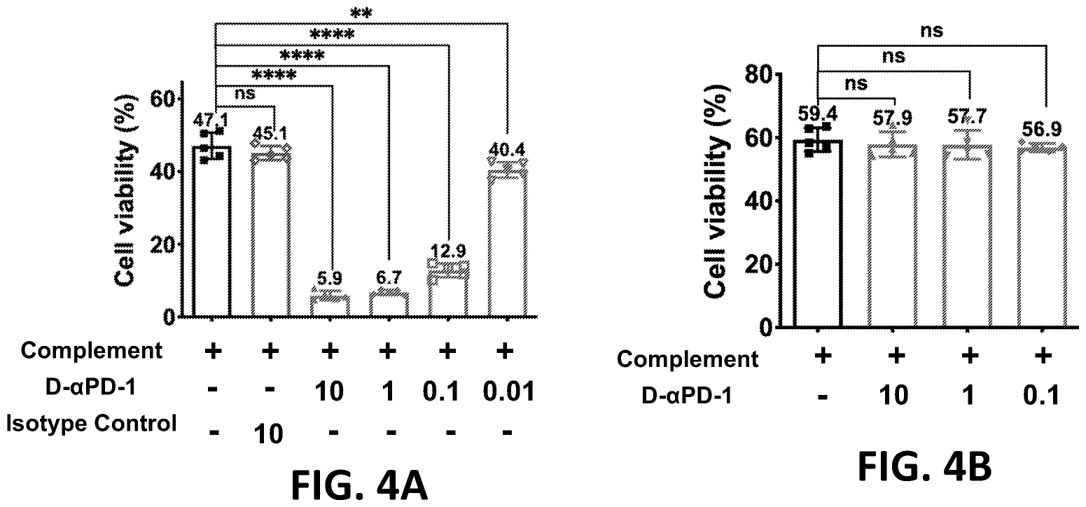

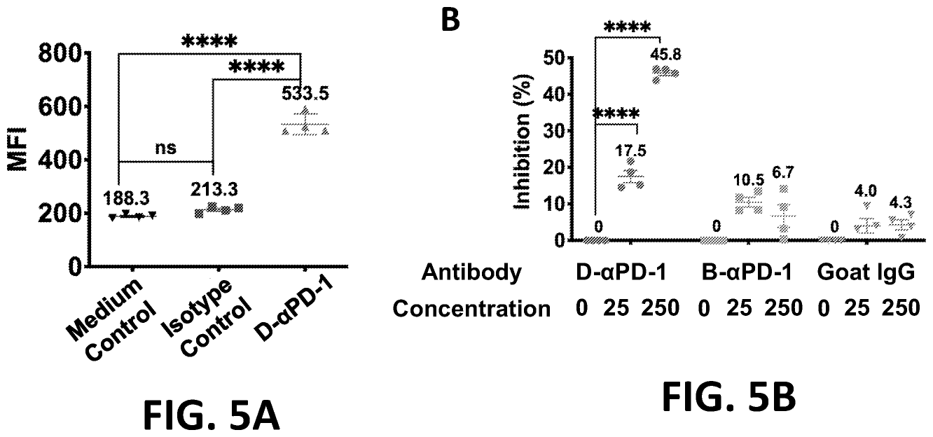

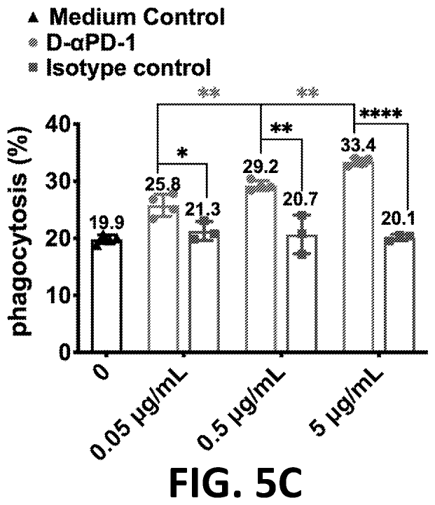

D-αPD-1 depletes PD-1⁺ cells through CDC and ADCP. The effector mechanisms that D-αPD-1 might utilize to deplete PD-1⁺ cells were assessed. The first examined mechanism was CDC, which is often employed by depleting antibodies. Here, target EL4 cells received six different treatments: complement, complement plus D-αPD-1 at 4 different concentrations, and complement plus an IgG2a isotype control. The viability of EL4 cells after treatment were quantified and compared among treatments (FIG. 4A). The complement shows a baseline toxicity to EL4 cells, resulting in a viability of 47%. However, the addition of D-αPD-1 drastically enhanced the toxicity to EL4 cells. The viability for EL4 cells that were treated with complement plus 10 μg/mL of D-αPD-1 decreased to one-eighth of the cells treated with complement alone (5.9% vs 47.1%, P<0.0001). In contrast, EL4 cells treated with the complement plus 10 μg/mL isotype control showed the similar viability as the cells treated with the complement alone (45.1% vs 47.1%. NS). These results show that D-αPD-1 is able to mediate CDC against PD-1⁺ cells. What further enhances this conclusion is the discovery that the effect of D-αPD-1 is concentration-dependent. When the concentration of D-αPD-1 used for the treatments increased from 0.01 μg/mL to 10 μg/mL, the viability of EL4 cells decreased from 40.4% to 5.9%. It is also noteworthy that the concentrations of D-αPD-1 in the range of 0.01 to 10 μg/mL caused significant reduction in viability as compared to the cells treated with the complement alone, which highlights the potency of D-αPD-1 in mediating CDC. The above D-αPD-1-mediated CDC is dependent on PD-1 on EL4 cells. When EL4 (PD-1^{KO}) cells were used to conduct the CDC assay, sharply different results emerged (FIG. 4B). The treatment of D-αPD-1 did not lower the cell viability compared with treatment of the complement alone. Further, the three concentrations of D-αPD-1 yielded the same viabilities. These results together show that PD-1 on EL4 cells is important for the D-αPD-1-mediated CDC.

ADCP was the next mechanism that was investigated. Before evaluating D-αPD-1-mediated ADCP by macrophages, the binding between D-αPD-1 and macrophages was investigated since the binding is the prerequisite of ADCP. Here, RAW 264.7 cells were incubated with Alexa-647-labeled D-αPD-1, isotype control, or the medium control. Then, the bound antibodies were analyzed by flow cytometry (FIG. 5A). Compared to cells in the medium control, RAW 264.7 cells incubated with D-αPD-1 had an MFI as of 533.5, which was approximately 3 times higher (P<0.0001). In contrast, isotype control treated cells had similar MFI as that of cells in the medium control (213.3 vs 188.3, NS). These results show that D-αPD-1 binds with RAW 264.7 macrophages.

Next, it was verified that the binding between D-αPD-1 and macrophages was mediated by mouse FcγRIV, a Fc receptor on macrophages that interacts with mouse IgG2a antibodies (Bruhns, P., Blood, 2012. 119(24); p. 5640-5649). This study was completed through a competitive assay. Specifically, RAW 264.7 cells that are FcγRIV-positive were preincubated with D-αPD-1, B-αPD-1 (a rat IgG), and a goat IgG control. Then, the treated RAW 264.7 samples were stained with a PE-labeled anti-mouse FcγRIV antibody. The binding of D-αPD-1, B-αPD-1, and the goat IgG to FcγRIV were measured by their capacity to inhibit the association of anti-mouse FcγRIV antibody with RAW 264.7 cells (FIG. 5B). D-αPD-1 showed a salient inhibition capacity, reducing the binding of anti-mouse FcγRIV antibody by 17.5% and 45.8% when used at 25 μg/mL and 250 μg/mL, respectively (P<0.0001). The treatment of B-αPD-1 and the goat IgG, on the other hand, showed slight inhibition, approximately 10% each. And the inhibition resulted from B-αPD-1 and goat IgG were not concentration-dependent, which may be attributed to non-specific interactions between B-αPD-1 and goat IgG with RAW 264.7 cells. These inhibition data, together with the binding results above, confirmed that D-αPD-1 is bound with macrophages and that such binding was through FcγRIV.

Lastly, ADCP was examined using CFSE-labeled EL4 cells as the target cells and RAW 264.7 cells as the effector cells. The two cell populations were mixed at 1:1 ratio and incubated with increasing concentrations of D-αPD-1 and an IgG2a isotype control, or the medium control. Phagocytized target cells by RAW 264.7 cells were quantified using flow cytometry (FIG. 5C). At the three antibody concentrations, D-αPD-1 resulted in a significantly higher percentage of phagocytosis as compared to the isotype control. For example, at the concentration of 5 μg/mL, the percentages of phagocytosis from the D-αPD-1 and isotype control treatments are 33.4% and 20.1%, respectively (P<0.0001). The promotion of phagocytosis by D-αPD-1 is dose-dependent. When mixed cells were incubated with 0.05. 0.5 and 5 μg/mL D-αPD-1 antibody, the percentages of phagocytosis were 25.8%, 29.2%, and 33.4% respectively. In contrast, such a dose-dependent trend does not exist in the isotype control treated cell samples. Indeed, the isotype controls treated cell samples has the same low phagocytosis as that of the medium control. Together, these data pointed to the notion that D-αPD-1 can promote ADCP of PD-1⁺ cells.

Discussion Described herein, is a depleting anti-PD-1 antibody, D-αPD-1. D-αPD-1 specifically binds with mouse PD-1⁺ cells and is able to utilize ADCP and CDC to phagocytose and ablate PD-1⁺ cells. D-αPD-1 induces the depletion of PD-1⁺ cells in vivo. The in vivo effect is strikingly potent since the treatment of D-αPD-1 effectively abolished PD-1⁺ target cells used in this experiment. These cells are transferred EL4 lymphoma cells and propagate robustly in mice if not eliminated. However, the treatment of D-αPD-1 is able to wipe out these transferred cells and keep transferred mice free from tumor growth and survive through the entire 80-day study.

A robust molecule or tool to deplete PD-1⁺ cells is desired for multiple reasons. First, PD-1⁺ lymphocytes play an important role in the progression of autoimmune diseases (Joller, N., et al., Immunol Rev, 2012. 248(1); p. 122-39; Salama, A. D., et al., J Exp Med, 2003. 198(1); p. 71-8; Okazaki, T., et al., Nat Immunol, 2013. 14(12); p. 1212-8; and Zhao. P., et al., Nat Biomed Eng. 2019. 3(4); p. 292-305). Thus, depletion of these lymphocytes in autoimmune disease patients can be used as a therapeutic strategy to treat autoimmune diseases, as evidenced by data in EAE and type-1 diabetes models (Zhao, P., et al., Nat Biomed Eng, 2019. 3(4); p. 292-305). Two additional advantages of this therapeutic strategy for autoimmune diseases are: first, the depletion covers both PD-1⁺ B cells and PD-1⁺ T cells so that it can abolish a wide and more comprehensive range of pathogenic immune cells in autoimmune diseases; second, the depletion affects activated lymphocytes and keeps the vast majority of lymphocytes, which are naive lymphocytes, so that patients received the depletion treatment still possess the normal lymphocyte reservoir and are able to defend against future infections and malignancy.

Beside autoimmune diseases, the depletion of PD-1⁺ cells may also find utility in cancer immunological research and therapy. First, PD-1⁺ tumor cells have been found in a wide range of cancer types and are believed to contribute to the resistance to the PD-1 immune checkpoint therapy and hyper-progression associated with the therapy (Ratner, L., et al., N Engl J Med. 2018. 378(20); p. 1947-1948; and Rauch. D. A., et al., Blood, 2019. 134(17); p. 1406-1414). Thus, depletion of these PD-1⁺ tumor cells can provide much needed solution for the resistance and the hyper progression. Second, in tumors, PD-1⁺ effector T cells, PD-1⁺ Tregs, and PD-1⁺ tumor cells in some cases (Xu-Monette, et al., Blood, 2018. 131(1); p. 68-83; Wang, X., et al., Proc Natl Acad Sci USA, 2020. 117(12); p. 6640-6650; Kleffel. S., et al., Cell, 2015. 162(6); p. 1242-56; Yao. H., et al., Front Immunol, 2018. 9: p. 1774; Du, S., et al., Oncoimmunology, 2018. 7(4); p, e1408747; and Schatton, T., et al., Cancer Res, 2010. 70(2); p. 697-708) create a PD-1 cell microenvironment. This environment is highly intertwined with the PD-1 immune checkpoint or the blockade of the checkpoint. The environment may dictate the outcome of the blockade therapy. On other hand, the blockade therapy itself may reshape the environment.

An immunotoxin that depletes PD-1⁺ cells to treat autoimmune diseases has been designed and developed (Zhao, P., et al., Nat Biomed Eng, 2019. 3(4); p. 292-305). As described herein, an alternative approach to deplete PD-1′ cells other than using an immunotoxin since immunotoxins cause safety concerns (Bera, T. K., et al., Leuk Res, 2014. 38(10); p. 1224-9) was designed and developed as described here. Different types of molecules, apart from immunotoxins, have been utilized for cell depletion, such as small molecules, antibodies, and antibody-drug-conjugates. Among these molecules, depleting antibodies are advantageous for their long plasma half-lives and plenteous preclinical and clinical information (Deligne, C., et al., Front Immunol, 2017. 8: p. 950). One clinically successful example is Rituximab, a depleting anti-CD20 antibody that is safely used to treat Non-Hodgkin's Lymphoma (NHL), Chronic Lymphocytic Leukemia (CLL), and Rheumatoid arthritis (RA) (McLaughlin, P., et al., J Clin Oncol, 1998. 16(8); p. 2825-33). Furthermore, two second generation depleting anti-CD20 antibodies ocrelizumab and ofatumumab are approved for the treatment on multiple sclerosis and CLL, respectively. And another second generation depleting anti-CD20 antibody veltuzumab is currently under phase I/II clinical study (Singh, V., D., et al., J Cancer Sci Ther, 2015. 7(11); p. 347-358). To fill the need to treat cancer and autoimmune diseases targeting αPD-1, depleting (D-αPD-1) antibodies were designed and developed. The C_{H} domains of D-αPD-1 is from mouse IgG2a, whose Fc fragment is able to bind with activating receptor FcγRIV with higher affinity compared with other antibody subtypes (Bruhns, P., Blood, 2012. 119(24); p. 5640-5649). FcγRIV is strictly expressed on monocytes, macrophages, and neutrophils, which are the main effector cells that are able to deplete cells ((Bruhns, P., Blood, 2012. 119(24); p. 5640-5649). The variable domains (V_{H} and V_{L}) of D-αPD-1 are designed based on B-αPD-1 (Zhao. P., et al., Nat Biomed Eng, 2019. 3(4); p. 292-305) which makes it possible for D-αPD-1 to recognize and bind with mouse PD-1. The combination of these constant and variable domains enables D-αPD-1 to specifically bind to and eliminate mouse PD-1⁺ cells.

When PD-1⁺ cells were incubated with D-αPD-1 at 4° C. and 37° C. cells at 37° C. accumulated more antibodies than cells at 4° C. The greater accumulation may have two causes: greater number of antibodies bind with the cells at higher temperatures, or antibodies, together with antigens, are internalized by cells at a higher temperature. It is likely that the later is true as evidence exists that PD-1 is internalized (Zhao. P., et al., Nat Biomed Eng, 2019. 3(4); p. 292-305; and Meng, X., et al., Nature, 2018. 564(7734); p. 130-135). For example, the PD-1 immunotoxin disclosed herein needs to reach the cytoplasm of PD-1⁺ cells to exert its cytotoxicity, and PD-1 immunotoxin has been found toxic to PD-1⁺ cells (Zhao, P., et al., Nat Biomed Eng, 2019. 3(4); p. 292-305). It is plausible that at least a fraction of D-αPD-1 that binds with PD-1 were internalized by PD-1⁺ cells. To further improve D-αPD-1, it is desirable to dampen internalization of the D-αPD-1/PD-1 complex. The functional design of D-αPD-1 is such that it remains on the cell surface for a sufficiently long time in order for effector cells and molecules to interact with the Fc of the antibody (Scott, A. M., et al, Nat Rev Cancer, 2012. 12(4); p. 278-87). The internalization of depleting antibodies could be modulated by altering antibody structure (Peng, L., et al., J Mol Biol, 2011. 413(2); p. 390-405) and using endocytosis inhibitors (Chew, H. Y., et al., Cell, 2020. 180(5); p. 895-914.e27)). Interestingly, internalization was also identified as one reason for the resistance to Rituximab (Roghanian, A., et al., Cancer Cell, 2015. 27(4); p. 473-88). Yet, Obinutuzumab, a second generation of anti-CD20 antibody, is not sensitive to internalization, although the reason for such a difference is still not clear (Lim, S. H., et al., Blood, 2011. 118(9); p. 2530-2540). In sum, reducing the internalization of the D-αPD-1/PD-1 complex is useful to boost the cell depletion effect of D-αPD-1.

Together, the results described herein show that, D-αPD-1 specifically binds to and depletes PD-1⁺ cells. The depleting capacity makes the antibody a useful and therapeutic strategy in both cancer and autoimmune diseases.

Materials and methods. Animals, cell lines and antibodies. Female C57BL/6 mice were purchased from The Jackson Laboratories. EL4 (ATCCR TIB-39™) cells were purchased from ATCC and were maintained in DMEM medium with 10% horse serum. EL4 (PD-1^{KO}) cells were generated and maintained in the same medium for EL4 (Zhao, P., et al., Nat Biomed Eng, 2019. 3(4); p. 292-305). Macrophage Raw 264.7 cells were purchased from ATCC and maintained in RPMI 1640 medium with 10% FBS. Expi293 expression system was purchased from ThermoFisher and applied following the manufacture instruction. PE anti-mouse IgG2a antibody (clone: m2a-15F8) was purchased from Ebioscience. APC anti-mouse CD3 (clone: 17A2). FITC anti-mouse CD3 (clone: 17A2), BV510 anti-mouse CD4 (clone: RM4-5), APC/Cyanine7 anti-mouse CD8 (clone: 53-6.7), PE anti-mouse TCR VB12 (clone: MR11-1), APC anti-mouse PD-1 (homemade), APC anti-mouse CD11b (clone: M1/70), PE anti-mouse PD-1 (clone: RMP1-14) and PE anti-mouse FcγRIV (clone: 9E9) were purchased from Biolegend.

Generation of expression vectors of D-αPD-1. The coding genes of the variable domains (V_{H} and V_{L}) of D-αPD-1 are based on a rat anti-mouse PD-1 antibody RMP1.14, and constant domains are from mouse IgG2a (Genbank ID: BAC44883.1). Genes encoding light chain and heavy chain were separately inserted into pcDNA3.4 expression vector and synthesized by Thermofisher. The sequences of these two construct genes were verified by DNA sequencing (Genewiz).

DNA gel electrophoresis. One μg heavy chain plasmid DNA was digested by 0.5 μL XbaI and 0.5 μL EcoRV, or 1 μg light chain plasmid DNA was digested by 0.5 μL SacI and 0.5 μL EcoRV with 1 μL rCutSmart buffer in a 10 μL reaction at 37° C. for 3 hours. Each sample with addition of 1 μL loading dye was loaded into the wells of a 1% agarose gel. The electrophoresis was run at 135 V for 30 minutes. The image of the gel was taken using the FluorChem FC2 imaging system (Alpha Innotech).

Protein expression and purification. B-αPD-1 was produced as previous report (Zhao, P., et al., Mol Pharm, 2017. 14(5); p. 1494-1500). Heavy and light chains of D-αPD-1 encoded on two separate plasmids were co-transfected into Expi293 cells at ratio of 1:2 (heavy chain:light chain). The transfected cells were cultured at 37° C. with 5% CO₂ for 6 days. Then the culture was collected and centrifuged. The supernatant was concentrated using Amicon ultra centrifugal filter units (30K). The antibody in the concentrated supernatant was purified through binding with Protein G beads (ThermoFisher). Antibody was eluted using 0.1 M Glyine (pH=2.8) and neutralized with IM Tris-HCl (pH=8.5). The yield, purity and integrity of the collected proteins were examined by both non-reducing and reducing SDS-PAGE analysis.

Evaluation of binding and uptake of D-αPD-1 by EL4 cells. For the evaluation of binding between D-αPD-1 and EL4 cells, 1×10⁵ EL4 cells were incubated with 0, 0.1, 1, and 10 μg/mL D-αPD-1 or control IgG2a at 4° C. for 30 minutes. EL4 cells were then stained with PE-anti-IgG2a and were analyzed by BD FACSCANTO II flow cytometer (BD Biosciences, San Jose, CA). For the evaluation of binding and uptake of D-αPD-1 by EL4 cells, D-αPD-1 was firstly labeled with Alexa Fluor 647 NHS Ester. Then 1×10⁵ EL4 cells were incubated with 0, 1, and 10 μg/mL labeled D-αPD-1 at 4° C. for binding only or 37° C. for binding and uptake for an hour. The EL4 cells were then analyzed using BD FACSCANTO II flow cytometer (BD Biosciences, San Jose, CA) and the mean fluorescence intensity (MFI) of the cells was reported.

In vivo EL4 cell depletion. 3×10⁶ of EL4 cells were transferred into C57BL/6 mice through tail vein IV injection at day 0. The transferred mice were randomly separated into 3 groups and were respectively treated with 200 μg D-αPD-1, B-αPD-1 or IgG2a at day 1 and day 10. Mice were then sacrificed at day 12 and cells from bone marrow were collected from mice. Cells were then stained with APC anti-mouse CD3 and PE anti-mouse PD-1 and analyzed using BD FACSCANTO II flow cytometer (BD Biosciences, San Jose, CA). The fraction of PD-1+ cells among T cells (CD3+ cells) were quantified to show the EL4 cell depletion.

EL4 tumor inhibition and EL4 cell detection. 2×10⁴ EL4 cells were transferred into C57BL/6 mice through tail vein IV injection at day 0. The transferred mice were randomly separated into 3 groups and were respectively treated with 200 μg D-αPD-1, IgG2a or PBS through IP injection at day 1. Their survival condition was observed through the whole study and median survival days were recorded.

2×10⁴ EL4 (PD-1^{KO}) cells were transferred into C57BL/6 mice through tail vein IV injection at day 0. The transferred mice were randomly separated into 2 groups and were treated with 200 μg D-αPD-1 or PBS through IP injection at day 1. The median survival days of these two groups were recorded. 2×10⁴ EL4 cells were transferred into C57BL/6 mice through tail vein IV injection at day 0. The transferred mice were randomly separated into 2 groups and were respectively treated with 200 μg D-αPD-1 or PBS through IP injection at day 1. Mice were sacrificed at several datapoints, and cells from blood and bone marrow were collected from mice. Cells were then stained with FITC anti-mouse CD3, BV510 anti-mouse CD4, APC/Cy7 anti-mouse CD8, PE anti-mouse TCR Vβ12 and APC anti-mouse PD-1, and analyzed using BD FACSCANTO II flow cytometer (BD Biosciences, San Jose, CA). EL4 cells were gated using CD4-CD8-TCR Vβ12+ phenotype markers (Meunier, M. C., et al., Blood, 2003. 101(2); p. 766-70). The fraction of EL4 cells among T cells (CD3+ cells) were quantified to show the EL4 tumor elimination.

In vitro ADCP assay. 1:1 ratio of 1×10⁵ macrophage Raw 264.7 cells and 1×10⁵ CFSE-labeled EL4 cells were co-cultured with 0, 0.05, 0.5, and 5 μg/mL antibodies at 37° C. with 5% CO₂ for 2 hours. The cells were washed and stained with APC anti-mouse CD11b at 4° C. for 30 minutes. Then the washed cells were analyzed using BD FACSCANTO II flow cytometer (BD Biosciences, San Jose, CA) to evaluate the phagocytosis of EL4 cells by macrophage Raw 264.7 cells. EL4 cells were gated by FITC and SSC signals. Macrophage cells were gated by APC and SSC signals. FITC and APC double positive cells represent the EL4 cells engulfed by Raw264.7 cells. The percentage of phagocytosis was defined as: Number of FITC and APC double positive cells/Total number of Raw 264.7 cells.

Evaluation of D-αPD-1 binding with macrophage Raw 264.7 cells. D-αPD-1 or IgG2a were firstly labeled with Alexa Fluor 647 NHS Ester. Then 1×10⁵ macrophage Raw 264.7 cells were co-cultured with 4 μg/mL Alexa-647-labeled antibodies at 4° C. for 30 minutes. Cells were then washed and analyzed using BD FACSCANTO II flow cytometer (BD Biosciences, San Jose, CA) to evaluate the binding between D-αPD-1 and macrophage Raw 264.7 cells.

Binding inhibition study with D-αPD-1. 1×10⁵ of macrophage Raw 264.7 cells were treated with D-αPD-1. B-αPD-1, and goat IgG at concentration of 0, 25, and 250 μg/mL at 4° C. for 30 minutes. Then the cells were stained with 2.5 μg/mL PE anti-mouse FcγRIV at 4° C. for 30 minutes. Cells were then analyzed using BD FACSCANTO II flow cytometer (BD Biosciences, San Jose, CA) to evaluate the binding between anti-FcγRIV and macrophage Raw 264.7 cells. Inhibition percentages of different groups were calculated based on the cells incubated without antibody treatment.

In vitro CDC assay. 5×10⁴ EL4 cells per well were co-cultured with 1/30 baby rabbit complement dilution and D-αPD-1 at concentration of 0, 0.01, 0.1, 1, and 10 μg/mL, or 10 μg/mL IgG2a as control at 37° C. for 3 hours. 5×10⁴ EL4 (PD-1^{KO}) per well were co-cultured with 1/30 baby rabbit complement dilution and 0, 0.1, 1, and 10 μg/mL D-αPD-1 at 37° C. for 3 hours. Then MTS (Promega) assay was then used for evaluation of EL4 cell viability (Zhao, P., et al., Nat Biomed Eng, 2019. 3(4); p. 292-305). In brief, cells after incubation were incubated with MTS/PMS reagents for 4 hours, then the OD₄₉₀ of each treated sample was measured. The cell viability was calculated based on the equation:

$$Cell viability ( \% ) = ( O {D}_{treated} - O {D}_{dead controi} ) / ( {OD}_{live control} - {OD}_{dead control} ) \times 100$$

OD_{treated}: OD₄₉₀ of D-αPD-1 or IgG2a treated cells.
OD_{dead control}: OD₄₉₀ of dead control (Triton X-100 treated cells)
OD_{live control}: OD₄₉₀ of the live control.

Statistical test. Unpaired two-sided t-test were used to compare data other than survival data. The survival curves are calculated from different treatment groups using the Kaplan-Meier estimation approach and compared their difference using log-rank test. The level of test significance was defined as p-value <0.05. The survival analyses were conducted using statistical software R (version 4.1.2).

1. An isolated antibody comprising a light chain variable region and a heavy chain variable region, wherein the light chain variable region comprises a complementarity determining region light chain 1 (CDRL1) amino acid sequence of SEQ ID NO: 4; a complementarity determining region light chain 2 (CDRL2) amino acid sequence of SEQ ID NO: 5; and a complementarity determining region light chain 3 (CDRL3) amino acid sequence of SEQ ID NO: 6; and wherein the heavy chain variable region comprises a complementarity determining region heavy chain 1 (CDRH1) amino acid sequence of SEQ ID NO: 1; a complementarity determining region heavy chain 2 (CDRH2) amino acid sequence of SEQ ID NO: 2; and a complementarity determining region heavy chain 3 (CDRH3) amino acid sequence of SEQ ID NO: 3.

2. The isolated antibody of claim 1, comprising a light chain variable region amino acid sequence of SEQ ID NO: 8, 10, 13, 14 or 15.

3. The isolated antibody of claim 1, comprising a heavy chain variable region amino acid sequence of SEQ ID NO: 7, 9, 11, or 12.

4. (canceled)

5. The isolated antibody of claim 1, wherein the light chain variable region has an amino acid sequence that is at least 90% identical to amino acid sequence of SEQ ID NO: 8, 10, 13, 14 or 15.

6. The isolated antibody of claim 1, wherein a heavy chain variable region has an amino acid sequence that is at least 90% identical to amino acid sequence of SEQ ID NO: 7, 9, 11, or 12.

7. The isolated antibody of claim 1, wherein the antibody is recombinantly engineered, chimerized, or humanized.

8. The isolated antibody of claim 1, wherein the antibody is a Fab, an Fab′, an F(ab′)2, a Fv, a scFv, a diabody or fragments thereof.

9.-12. (canceled)

13. The isolated antibody of claim 1,
wherein the antibody is a monoclonal antibody.

14. (canceled)

15. The isolated antibody of claim 1,
wherein the antibody is linked to a detectable label.

16.-18. (canceled)

19. A composition comprising the isolated antibody of claim 1, and at least one pharmaceutically acceptable carrier or diluent.

20. (canceled)

21. A method of treating an autoimmune disease in a subject, the method comprising administering to the subject a therapeutically effective amount of the isolated antibody of claim 1.

22. The method of claim 21, wherein the autoimmune disease is a T-cell mediated autoimmune disease.

23. The method of claim 21, wherein the autoimmune disease is multiple sclerosis, type-1 diabetes, systemic lupus erythematosus, or rheumatoid arthritis.

24. A method of depleting αPD-1 positive cells in a subject, the method comprising administering to the subject a therapeutically effective amount of an isolated antibody of claim 1.

25. The method of claim 24, wherein the αPD-1 positive cells are malignant.

26. The method of claim 24, wherein the αPD-1 positive cells are lymphoctyes or autoreactive immune cells.

27. (canceled)

28. (canceled)

29. A method of treating lymphoma in a subject, the method comprising administering to the subject a therapeutically effective amount of the isolated antibody of claim 1.

30. A method of treating a cancer in a subject, the method comprising administering to the subject a therapeutically effective amount of the isolated antibody of claim 1.

31. A method of treating metastatic cancer in a subject or preventing metastasis in a subject having cancer at risk for metastasis, the method comprising administering to the subject a therapeutically effective amount of the isolated antibody of claim 1.

32. The method of claim 30, wherein the cancer is a cancer of breast, colon, lymphatic system, pancreas, lung, skin, esophagus, bladder, head and neck, and stomach.

33.-38. (canceled)


SEQUENCE LISTING (15 sequences)

SEQ ID NO: 1 - protein, 6 aa, synthetic construct
SSYRWN

SEQ ID NO: 2 - protein, 16 aa, synthetic construct
YINSAGISNYNPSLKR

SEQ ID NO: 3 - protein, 11 aa, synthetic construct
SDNMGTTPFTY

SEQ ID NO: 4 - protein, 16 aa, synthetic construct
RSSKSLLYSDGKTYLN

SEQ ID NO: 5 - protein, 7 aa, synthetic construct
WMSTRAS

SEQ ID NO: 6 - protein, 8 aa, synthetic construct
QQGLEFPT

SEQ ID NO: 7 - protein, 120 aa, synthetic construct
EVQLQESGPGLVKPSQSLSLTCSVTGYSITSSYRWNWIRKFPGNCLEWMGYINSAGISNYNPSLKRRISITRDTSKNQFFLQVNSVTTEDAATYYCARSDNMGTTPFTYWGQGTLVTVSS

SEQ ID NO: 8 - protein, 111 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGCGTKLELK

SEQ ID NO: 9 - protein, 120 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 10 - protein, 111 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK

SEQ ID NO: 11 - protein, 468 aa, synthetic construct
MRMLVLLYLLTALPGILSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 450 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 238 aa, synthetic construct
MRCSLQFLGLLVLWIPGLNGDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 219 aa, synthetic construct
GDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC